(19) United States
(12) Patent Application Publication
(10) Pub. No.: US 2009/0123477 A1
(43) Pub. Date: May 14, 2009

(54) Antibodies

(76) Inventors: Thomas Hanke, Wurzburg (DE); Frank Horling, Wurzburg (DE); Martin Trischler, Wurzburg (DE)

Correspondence Address: MAYER & WILLIAMS PC, 251 NORTH AVENUE WEST, 2ND FLOOR, WESTFIELD, NJ, 07090, US

(21) Appl. No.: 11/318,352

(22) Filed: Dec. 23, 2005

(30) Foreign Application Priority Data
Dec. 23, 2004 (DE) DE102004063494.7

(51) Int. Cl. A61K 39/395; C07K 16/18; C07K 7/00; C07K 14/00; C12P 21/04; C12N 15/11; C12N 15/00; C12N 5/06

(52) U.S. Cl. 424/144.1; 530/388.22; 530/387.3; 530/328; 530/326; 530/327; 530/329; 530/350; 536/23.1; 435/320.1; 435/325; 435/69.1

(57) ABSTRACT

The invention concerns an isolated monoclonal antibody, which is specific and agonistic for CTLA-4, whereby the antibody does not bind to the C″D loop of CTLA-4.

### FIELD OF THE INVENTION

The invention concerns an antibody, which is specific for CTLA-4, a pharmaceutical compound containing such an antibody, nucleic acid encoding such antibodies, vectors containing such antibodies, cells transfixed with such vectors, applications of such antibodies, methods for the production of such antibodies and methods for the production of a pharmaceutical compound containing such antibodies.

### BACKGROUND OF THE INVENTION AND STATE OF THE ART

T lymphocytes (T cells) are the main agents of a highly efficient immune response that protects the human body against penetrating pathogens, such as bacteria and viruses. They regulate the molecular interaction between different cellular components of the immune system, such as dendritic cells, B cells, macrophages or other T cells, and carry out important effector functions themselves, such as the destruction of virus-infected cells or tumor cells. This means that they take up a key position in initiating and coordinating an immune response.

Highly active molecules called T cell antigen receptors (TCR) located on the cell surface give each T cell an identity and give them the ability to specifically recognise antigens presented by molecules of the major histocompatibility complex (MHC). Additional cell surface receptors of the ‘CD’ type regulate the method and type of T cell response, which is initiated by antigen-related stimulation of the TCR. Thus the TCR dictates the specific nature of an immune response, whilst the CD receptors control the scope and quality of the T cell response. Under physiological conditions, a combination of signals from TCR and at least one further CD receptor is required for the complete activation of T cells, which is particularly characterised by proliferation and cytokine production. This process is called ‘co-stimulation’. The most important co-stimulating CD molecule on resting human T cells is the CD28 molecule.

In order to avoid an overreaction of the immune system, which would lead to an uncontrolled and hence dangerous propagation of lymphocytes and a massive production of inflammatory cytokines, it is necessary to effectively switch off the activation of T cells. This task is achieved by the combination of a number of immunological control mechanisms. For this purpose, inhibiting cell surface receptors, such as the ‘cytotoxic T lymphocyte antigen-4’ (CTLA-4) molecule, which will be explained in greater detail later on, play a particularly important role.

In the development of autoimmune diseases, such as rheumatoid arthritis, type I diabetes, multiple sclerosis, colitis or psoriasis, as well as the development of allergies, an uncontrolled response of T lymphocytes to autologous structures and/or external antigens plays an important role. It is therefore quite possible that an initial overactivation of T cells, a missing inhibition of autoreactive T cells or a deficiency in the number and/or function of regulatory T cells has a causal connection with these diseases. Also in the case of allogenous organ transplantations, i.e. transplantations between individuals that are not HLA identical, an activation of the T cells of the recipient is not wanted since the activation of T cells is the main cause for chronic rejection reaction due to the recognition of alloantigen.

Current therapy concepts for suppressing the T cell response aim at the non-antigen-specific suppression of the activity of both harmful as well as useful T cells through the use of ‘nonspecific’ immune suppressants. This means that therapeutic effects are often accompanied by serious side effects.

CTLA-4 (CD152) is a member of the immunoglobulin superfamily and is structurally the nearest relative of CD28 (Lenschow D J, Walunas T L, Bluestone J A, CD28/B7 system of T cell costimulation. Annu Rev Immunol, 1996. 14:233-58). But in contrast to CD28, the physiological function of CTLA-4 is not the promotion but the inhibition of T cell activation. CTLA-4 is very weakly expressed on resting T cells and strongly on the cell surface of activated and regulatory T cells. The binding of CTLA-4 to its natural ligands B7-1 (CD80) and B7-2 (CD86), which are expressed by antigen-presenting cells (APC), leads to switching the T cell proliferation off and suppressing the cytokine expression (Egen J G, Kuhns M S, Allison J P, CTLA-4: new insights into its biological function and use in tumour immunotherapy. Nat Immunol, 2002. 3(7):611-8). The inhibiting function of CTLA-4 on the surface of T cells was initially demonstrated with the help of immobilised monoclonal antibodies specifically for the CTLA-4 molecule of the mouse (Walunas T L, Lenschow D J, Bakker C Y, Linsley P S, Freeman G J, Green J M, Thompson C B, Bluestone J A, CTLA-4 can function as a negative regulator of T cell activation, Immunity, 1994. 1(5):405-13) and humans (Blair P J, Riley J L, Levine B L, Lee K P, Craighead N, Francomano T, Perfetto S J, Gray G S, Carreno B M, June C H, CTLA-4 litigation delivers a unique signal to resting human CD4 T cells that inhibits interleukin-2 secretion but allows Bcl-X(L) induction, J Immunol, 1998. 160(1):12-5) and could be proved through the phenotype of mice in which the CTLA-4 gene was deliberately deactivated through homologous recombination. These animals died quickly from a lymphoproliferating disease, which is characterised by an uncontrolled activation of T cells (Tivol E A, Borriello F, Schweitzer A N, Lynch W P, Bluestone J A, Sharpe A H, Loss of CTLA-4 leads to massive lymphoproliferation and fatal multiorgan tissue destruction, revealing a critical negative regulatory role of CTLA-4. Immunity, 1995. 3(5): 541-7, as well as Waterhouse P, Penninger J M, Timms E, Wakeham A, Shahinian A, Lee K P, Thompson C B, Griesser H, Mak T W, Lymphoproliferative disorders with early lethality in mice deficient in Ctla-4. Science, 1995. 270(5238):985-8).

Conversely, these results suggest that CTLA-4 blockade reinforces the activation of T cells in vivo. In line with this, blocking, i.e. antagonistic, anti-CTLA-4 antibodies potentised an antitumour response (Chambers C A, Allison J P, Costimulation in T cell responses. Curr Opin Immunol, 1997. 9(3):396-404), but also induce autoimmunity (Luhder F, Hoglund P, Allison J P, Benoist C, Mathis D, Cytotoxic T lymphocyte-associated antigen 4 (CTLA-4) regulates the unfolding of autoimmune diabetes. J Exp Med, 1998. 187(3):427-32). These findings, which had first been gained in the mouse system, could also be confirmed in humans in the first clinical trials. For example, after administering blocking antihuman CTLA-4 antibodies, individual cases of patients with metastasizing melanoma experienced a (partial) remission (Hodi F S, Mihm M C, Soiffer R J, Haluska F G, Butler M, Seiden M V, Davis T, Henry-Spires R, MacRae S, Willman A, Padera R, Jaklitsch M T, Shankar S, Chen T C, Korman A, Allison J P, Dranoff G, Biologic activity of cytotoxic T lymphocyte-associated antigen 4 antibody blockade in previously vaccinated metastatic melanoma and ovarian carcinoma patients, Proc Natl Acad Sci USA, 2003. 100(8):4712-7). At the same time, clinical indications for autoimmunity were found in a large proportion of treated patients (Phan G Q, Yang J C, Sherry R M, Hwu P, Topalian S L, Schwartzentruber D J, Restifo N P, Haworth L R, Seipp C A, Freezer L J, Morton K E, Mavroukakis S A, Duray P H, Steinberg S M, Allison J P, Davis T A, Rosenberg S A, Cancer regression and autoimmunity induced by cytotoxic T lymphocyte-associated antigen 4 blockade in patients with metastatic melanoma, Proc Natl Adad Sci USA, 2003. 100(14):8372-7).

A polymorphism in the CTLA-4 gene leading to a reduced expression and functionality of the CTLA-4 protein correlates with an increased probability of people falling ill with autoimmune diseases such as rheumatoid arthritis (Seidl C, Donner H, Fischer B, Usadel K H, Seifried E, Kaltwasser J P, Badenhoop K, CTLA4 codon 17 dimorphism in patients with rheumatoid arthritis. Tissue Antigens, 1998. Jan; 51(1):62-6), multiple sclerosis (Harbo H F, Celius E G, Vartdal F, Spurkland A, CTLA4 promoter and exon 1 dimorphisms in multiple sclerosis. Tissue Antigens, 1999; 53(1):106-10) or type I diabetes (Donner H, Rau H, Walfish P G, Braun J, Siegmund T, Finke R, Herwig J, Usadel K H, Badenhoop K, CTLA4 alanine-17 confers genetic susceptibility to Graves' disease and to type 1 diabetes mellitus. J Clin Endocrinal Metab, 1997. 82(1):143-6).

In contrast to the reinforcement of a T cell response with the blocking/antagonistic anti-CTLA-4 antibodies described above, agonistic anti-CTLA-4 antibodies should have an immunosuppressive effect. However, up to now it was only possible to demonstrate that convincingly for artificially immobilised antibodies. Thus the transmembrane expression of a ‘single-chain’ anti-CTLA-4 antibody on artificial APC created in gene technology reduced the TCR-induced proliferation and Interleukin-2 dissemination of T cells (Griffin M D, Hong D K, Holman P O, Lee K M, Whitters M J, O'Herrin S M, Fallarino F, Collins M, Segal D M, Gajewski T F, Kranz D M, Bluestone J A, Blockade of T cell activation using a surface-linked single-chain antibody to CTLA-4 (CD152). J Immunol, 2000. 164(9):4433-42). The fact that, in this experimental approach, not only pre-activated but also resting T cells were inhibited shows that an important function of CTLA-4 is the early suppression of the TCR signal. Similar results were obtained by Brunner et al. (Brunner M C, Chambers C A, Chan F K, Hanke J, Winoto A, Allison J P, CTLA-4-Mediated inhibition of early events of T cell proliferation. J Immunol, 1999. 162(10):5813-20) in the analysis of CTLA-4 signal paths in naïve T cells.

The transmembrane expression of a single-chain anti-CTLA-4 antibody on allogenous tumour cells led to a reduction of the T-cell-conveyed elimination of these tumour cells in mice (Hwang K W, Sweatt W B, Brown I E, Blank C, Gajewski T F, Bluestone J A, Alegre M L, Cutting edge: targeted ligation of CTLA-4 in vivo by membrane-bound anti-CTLA-4 antibody prevents rejection of allogeneic cells. J Immunol, 2002. 169(2):633-7). These results showed that an immunological anti-tumour response or the rejection of allogeneic organ transplants can be suppressed through efficient crosslinking of CTLA-4. However, up to now this type of targeted suppression of T cell activation through CTLA-4 ligation in vivo could only be achieved with membrane-bound anti-CTLA-4 antibody constructs or with the natural membrane-based ligands. Up to now, a corresponding suppression of the T cell response in the animal through soluble anti-CTLA-4 antibodies has not been described. What has been described is the in vitro induction of apoptosis in pre-activated T cells by an CTLA-4 antibody with specificity for the C″D loop of the extracellular domain of CTLA-4 (Gribben J G, Freeman G J, Boussiotis V A, Rennert P, Jellis C L, Greenfield E, Barber M, Restivo V A Jr, X Ke, Gray G S, Nadler L M, CTLA-4 mediates antigen-specific apoptosis of human T cells. Proc Natl Acad Sci USA, 1995. 92(3):811-5).

In conclusion, the findings so far show an inhibiting function of CTLA-4 on T cells; however, it is not yet fully clear how this mechanism is affected. The following mechanism, which are not mutually exclusive, are discussed: i) suppression of the activating TCR and/or CD28 signal path, ii) competition of the CD28-induced costimulation through higher affinity to CD80 and CD86, iii) increasing the threshold value of T cell activation, iv) attenuation of the T cell expansion and/or v) activation of regulatory cells and connected with that indirect suppression of conventional T cells.

In the above-mentioned indications, a selective inactivation of T cells through the stimulation of the inhibiting function of CTLA-4, which is well tolerated by the organism, is desirable.

### THE TECHNICAL PROBLEM OF THE INVENTION

Therefore the invention is based on the technical problem of stating substances and pharmaceutical compounds that are capable of stimulating the inhibiting function of CTLA-4.

### Basic Characteristics of the Invention and Preferred Forms of Application.

To solve this technical problem, the invention teaches an isolated monoclonal antibody, which is specific and agonistic for CTLA-4, whereby the heavy chain of the antibody contains a sequence selected from the group consisting of (Seq.-ID): “22, 23, 24, 25, 26, 27, 28, 29, and 32”. The light chain of the antibody can contain a sequence that has been selected from the group consisting of (Seq.-ID): “33, 34, 35, 36, 37 and 38”.

The preference is for an antibody in accordance with the invention with a heavy chain containing a sequence in accordance with Seq.-ID 27, 28 or 29, preferably containing or consisting of the sequence in accordance with Seq.-ID 30 or 32, as well as with a light chain containing a sequence in accordance with Seq.-ID 36 or 37, preferably containing or consisting of a sequence in accordance with Seq.-ID 38.

Special antibodies with the above general structure are the antibodies TGN2122.H and TGN2422.H described below.

In addition, the invention teaches an isolated monoclonal antibody, which is specific and agonistic for CTLA-4, whereby the heavy chain of the antibody contains a sequence which is selected from the group consisting of (Seq.-ID): “43, 44, 45, 46, 47, 48, 49, 50, 51 and 53”. The light chain of the antibody can contain a sequence which is selected from the group consisting of (Seq.-ID): “54, 55, 56, 57, 58 and 59”.

The preference is an antibody, which is also in accordance with the invention, with a heavy chain containing a sequence in accordance with Seq.-ID 48, 49 or 50, preferably containing or consisting of a sequence in accordance with Seq.-ID 51 or 53 and with a light chain containing a sequence in accordance with Seq.-ID 57 or 58, preferably containing or consisting of a sequence in accordance with Seq.-ID 59.

Special antibodies with the above general structure are the antibodies TGN2122.C and TGN2422.C described below.

The above-mentioned antibodies are humanised antibodies. Since the antibodies are already humanised, a humanisation, as described below for further variants of antibodies covered by the invention, is not required. The antibody may, but does not have to bind to the C″D loop of CTLA-4. It may also be an antibody that does not bind to this loop. With respect to all further forms and applications as well as other details and explanations, the subsequent explanations for a further variant of the invention apply analogously and in full.

Finally, the invention teaches an isolated monoclonal antibody, which is specific and agonistic for CTLA-4, whereby the antibody does not bind to a partial CTLA-4 sequence in accordance with Seq.-ID 1. The sequence in accordance with Seq.-ID 1 is the C″D loop of the CTLA-4. Put another way, the antibody covered by the invention binds to other areas of the CTLA-4 molecule than the C″D loop. The invention is based on the finding that an agonistic stimulation of CTLA-4, i.e. inducing the inhibiting activity of CTLA-4 in vivo, is a reasonable therapeutic concept for autoimmune diseases or transplants, and provides suitable substances in the form of antibodies or fragments thereof for this purpose.

Antibodies covered by the invention contain preferably at least one of the sequences in accordance with Seq.-ID 2 to Seq.-ID 7 or Seq.-ID 8 to Seq.-ID 13. These sequences are the CDRs of the variable areas of a heavy and a light chain; please also refer to table 2.

Preferably an antibody covered by the invention is humanised. This can be accomplished using the usual methods, for example by chimaerising a specific monoclonal mouse antibody against human CTLA-4 in such a way that the constant areas are replaced by human constant areas or constant areas tolerated by human organisms. What is important is that preferably all CDRs in accordance with table 2 are retained, including their spatial arrangement to each other. Possible bases for the humanisation can be, for example, monoclonal antibodies containing at least one, but preferably all, sequences in accordance with Seq.-ID 2 to Seq.-ID 7 or Seq.-ID 8 to Seq.-ID 13, for example one of the sequences in accordance with Seq.-ID 14 to 17. In concrete situations, antibodies covered by the invention can also contain one of the sequences in accordance with Seq.-ID 18 to 21. Suitable realised examples of antibodies forming a basis for humanisation are the antibodies 4.8H10H5 and 4.3F6B5 describe in detail below. It is possible to make humanised antibodies from these using the usual methods of gene technology, for example by applying gene technological humanisation strategies.

In the context of the invention, the term antibody comprises the explicitly revealed structures as well as functionally equivalent antibodies, which have been modified using e.g. chimaerisation, humanisation, or de-immunisation (cutting out T cell epitopes from the human antibody that causes undesirable immune reactions), as well as specific fragments of the light and/or the heavy chain of the variable area of the antibodies of the type described above. The average professional in this field should be familiar with the production/cultivation of such antibodies with specified immunogens; therefore this does not have to be explained in detail.

The invention also concerns an isolated protein or peptide containing at least one of the sequences Seq.-ID 2 to 13, in particular one of the sequences Seq.-ID 14 to 17 or Seq.-ID 18 to 21, or one of the sequences Seq.-ID 22, 23, 24, 25, 26, 27, 28, 29, 30, 31, 32, 33, 34, 35, 36, 37, 38 or 39, in particular one of the sequences Seq.-ID 27, 28, 29, 30, 32, 36, 37 or 38, or one of the sequences Seq.-ID 43, 44, 45, 46, 47, 48, 49, 50, 51, 52, 53, 54, 55, 56, 57, 58, 59 or 60, in particular one of the sequences Seq.-ID 48, 49, 50, 51, 53, 57, 58 or 59, or consisting of one of the named sequences an isolated nucleic acid encoding for one such protein or peptide or for a light chain and/or a heavy chain of an antibody covered by the invention, an isolated vector containing such a nucleic acid, and an isolated cell, which is transfixed with such a vector. All the above items are suitable for the production or construction of antibodies in accordance with the invention.

An antibody in accordance with the invention or a protein or peptide in accordance with the invention should preferably be soluble in water, in particular in physiological salt solution, i.e. not artificially cross-linked. Also, an antibody in accordance with the invention is superagonistic, i.e. it stimulates the physiological activity of the T cell inhibiting receptor CTLA-4.

In addition, the invention concerns a pharmaceutical compound containing a monoclonal antibody in accordance with the invention and/or a protein or peptide in accordance with the invention as well as optionally at least one physiologically compatible carrier substance and/or agent, which will be explained in detail later on. It can be obtained by mixing these components, whereby the active substance is used in a physiologically effective dosis. This dosis can easily be determined with cells in in-vitro trials as well as with animal trials in the usual way. Such a pharmaceutical compound is suitable for prophylactic or therapeutic treatment of a disease or a condition from the group comprising “rheumatoid arthritis, type I diabetes, multiple sclerosis, systemic lupus erythematodes, psoriasis, ulcerative colitis, morbus crohn, allergies, rejection of allogenous organ transplants, in particular organ transplants of the following organs: heart, kidney, liver, pancreas, lung, bone marrow, and ‘Graft-Versus-Host’ disease”. To that extent, the invention also comprises a process for prophylaxis and/or treatment of one of the above diseases, whereby the patient is administered the pharmaceutical compound in a suitable dosis.

The galenic preparation of a pharmaceutical compound in accordance with the invention can be made in the usual way. Possible counter-ions for ionic compounds are for example Na⁺, K⁺, Li⁺ or cyclohexylammonium. Suitable solid or liquid galenic forms of preparations are for example granules, powder, coated tablets, tablets, (Micro) capsules, suppositories, syrups, juices, suspensions, emulsions, drops or solutions for injection (i.v., i.p., i.m., s.c.) or atomisation (aerosols), transdermal systems as well as preparations with protracted release of the active substance, for the production of which the usual auxiliary substances are used, such as carrier substances, blasting agents, binding agents, coating materials, swelling agents, lubricants, flavourings, sweeteners and solubilisers. Possible excipients are magnesium carbonate, titanium dioxide, lactose, mannite and other sugars, talcum, milk protein, gelatine, starch, cellulose and its derivatives, animal and vegetable oils such as cod liver oil, oil of sunflowers, peanuts or sesame, polyethyleneglycols and solvents such as sterile water and monohydric or poly hydric alcohols, for example glycerine.

In addition, the invention concerns a process for the production of a monoclonal antibody in accordance with the invention, in which a nucleic acid in accordance with the invention is entered into a vector, whereby a cell is transfixed with the help of the vector, whereby the transfixed cell is cultivated, whereby an excess of the cultivated cell is cut off or whereby the cultivated cell is lysed and the lysate is obtained, and whereby the monoclonal antibodies are separated from the cut off excess or the lysate.

Hereinafter, the invention is explained in greater detail, using examples presenting just different product forms.

### EXAMPLE 1

### Antibodies in Accordance with the Invention and Reference Antibodies

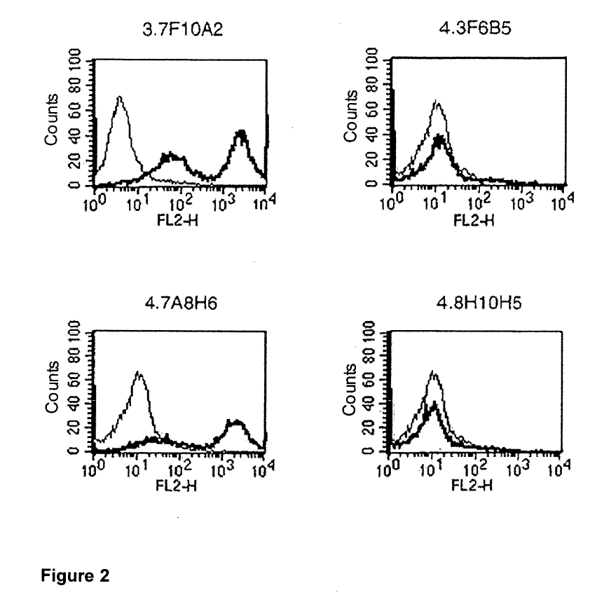

Table 1 shows the binding characteristics of 4 new anti-CTLA-4 antibodies, of which 2 do not bind to the C″D loop of CTLA-4 (4.3F6B5 and 4.8H10H5) and 2 that bind to it (3.7F10A2 and 4.7A8H6). The latter are reference antibodies and are not subject to this invention. Subject of the investigation was the specificity of the antibodies for human CTLA-4, both on transfixed Jurkat E6.1 cells as well as ex vivo activated human PBMCs (peripheral blood mononuclear cells). The cross reactivity against rat CTLA-4 was demonstrated with a transfixed BW cell line carrying the extracellular domain of rat CTLA-4 on the surface. Likewise the cross reactivity against the closely related T cell receptors CD28 and ICOS on transfixed Jurkat E6.1/L929 cells was eliminated. The binding or non-binding to the lateral C″D loop structure is illustrated in detail in FIG. 2. The thick curves represent CTLA-4 and the thin curves represent the isotype control.

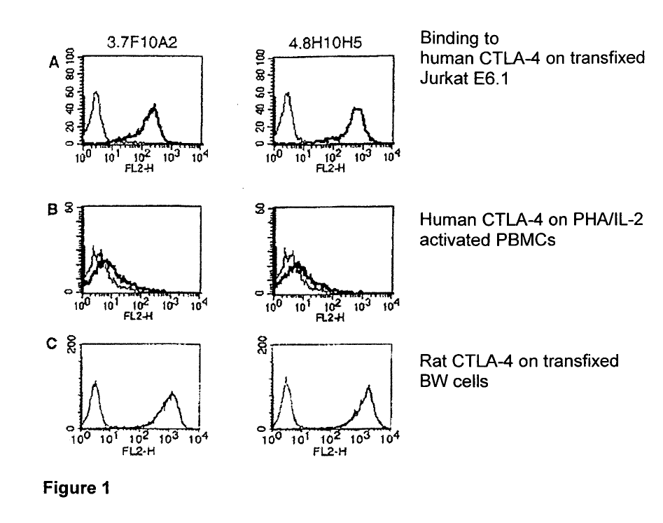

FIG. 1 shows examples of the most important binding characteristics of the anti-CTLA-4 antibody 4.8H10H5 in accordance with the invention as well as the reference antibody 3.7F10A2. (A) For the identification of CTLA-4 specific antibodies, transfixed Jurkat E6.1 cells were used that carry a chimaerous CTLA-4/CD28 receptor on their surface. This consists of the extracellular domain of human CTLA-4, which causes the specificity of the antibodies, and the transmembrane and intracellular domain of mouse CD28. The CD28 part of the receptor ensures a stable surface expression of the chimaerous receptor. The diagram shows the binding of the antibodies to transfixed cells (thick curve) in comparison to the binding to non-transfixed cells (thin curve). (B) The specificity of the antibodies for CTLA-4 was confirmed with human PBMCs, which before had been stimulated ex vivo with PHA/IL-2. In resting cells, the localisation of CTLA-4 is primarily intracellular and will not come to the surface until after activation. The thick curve shows the binding of the antibodies, the thin curve the binding of the isotype control to activated human PBMCs. (C) With a view to a possible use of the antibodies in animal models, the cross-reactivity against rat CTLA-4 was demonstrated. For this purpose, transfixed BW cells were used that carry a chimaerous human CTLA-4/mouse CD28 receptor on their surface. The diagram shows the binding of the antibody to transfixed cells (thick curve) and to non-transfixed cells (thin curve).

FIG. 2 shows the nonexisting specificity of 2 of the 4 anti-CTLA-4 antibodies from table 1 for the C″D loop. Surprisingly it turned out that the two antibodies 4.3F6B5 and 4.8H10H5 covered by the invention, which showed agonistic activity in functional assays (see example 2) are not specific for the human C″D loop. Jurkat E6.1 were used for this, which express a chimaerous extracellular domain of CTLA-4 on the surface: this chimaer consists of the murine receptor, in which the C″D loop was replaced for the corresponding human sequence, presented by the amino acids Pos 68-83. The binding of the antibodies 3.7F10A2 and 4.7A8H6 to the C″D loop shows that the construct was expressed efficiently. For the antibodies 3.7F10A2 and 4.7A8H6 this amino acid sequence is sufficient for binding (thick line, thin curve: isotype control). For the antibodies 4.8H10H5 and 4.3F6B5 this sequence is not sufficient for binding.

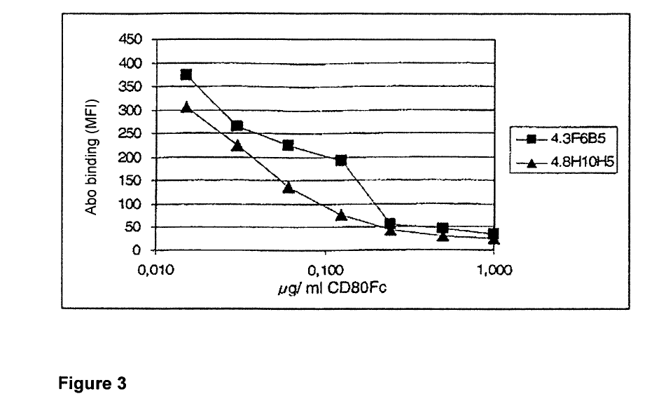

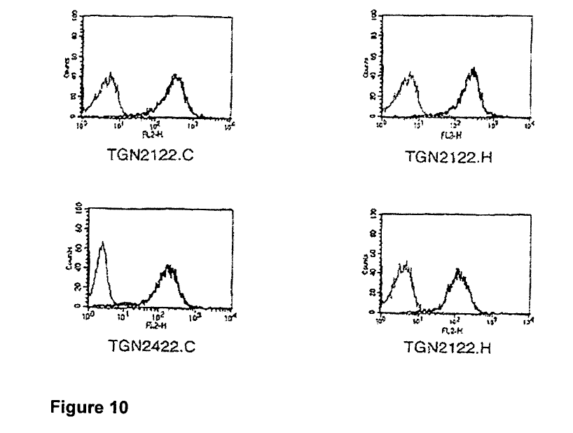

FIG. 3 shows that the binding of the anti-CTLA-4 antibodies 4.8H10H5 and 4.3F6B5 to CTLA-4 can be competed by adding recombinant CD80. The result suggests that 4.8H10H5 and 4.3F6B5 bind in the proximity of the binding location for CD80, i.e. the MYPPPY loop and not to the C″D loop. The aim of the experiment was to further localise the binding characteristics of the antibodies. Jurkat E6.1 cells, which carry the extracellular domain on their surface, were incubated with an increasing concentration of CD80Fc protein and 1 μg/ml CTLA-4 specific antibodies. The co-incubation of recombinant protein and antibodies leads to a replacement of the binding of the antibodies 4.8H10H5 and 4.3F6B5 to the extracellular domain of CTLA-4. Experiments as described in connection with FIG. 1 were also carried out for the antibodies TGN2122.C, TGN2422.C, TGN2122.H and TGN2422.H. FIG. 10 shows the binding of the humanised antibodies to the extracellular domain of human CTLA-4. The figure shows the binding of the humanised antibodies to transfixed cells (thick curve) compared to the binding of the isotype control (thin curve) to the same cells. The FACS analysis shows that the specificity of the antibodies for human CTLA-4 remains intact during the humanising process.

Table 2 contains the sequences of the 4.8H10H5 and 4.3F6B5 antibodies covered by the invention, with a division into heavy and light chains, with the boundary between the variable areas and the constant areas being marked. The sequences were determined with the help of RT-PCR and/or protein sequencing (Edman Abbau).

Table 3 contains sequences of the heavy chain of antibody TGN2122.H. Table 4 contains the nucleic acid encoding for the heavy chain. Table 5 contains sequences of the heavy chain of the antibody TGN2422.H. Table 6 shows the nucleic acid encoding for the heavy chain. Table 7 shows sequences of the light chain for both antibodies TGN2122.H and TGN2422.H. Table 8 shows the nucleic acid encoding for the light chain.

Table 9 shows sequences of the heavy chain of the antibody TGN2122.C. Table 10 shows the nucleic acid encoding for the heavy chain. Table 11 shows sequences of the heavy chain of the antibody TGN2422.C. Table 12 shows the nucleic acid encoding for the heavy chain. Table 13 shows sequences of the light chain for both antibodies TGN2122.C and TGN2422.C. Table 14 shows the nucleic acid encoding for the light chain.

In the case of the sequences Seq.-ID 31, 39, 52 and 60 we are dealing with leader peptides, which are not included in the respective mature chains. Therefore antibodies are preferred that do not contain these sequences.

The antibodies TGN2122.C (isotype IgG1) and TGN2422.C (isotype IgG4) were obtained by humanisation from the mouse antibody 4.3F6B5. The antibodies TGN2122.H (isotype IgG1) and TGN2422.H (isotype IgG4) were obtained from the mouse antibody 4.8H10H5.

### EXAMPLE 2

### The Effect of Antibodies in Accordance with the Invention Compared to a Reference Antibody Binding to the C″D Loop, as Well as Commercially Obtainable Anti-CTLA-4 Antibodies.

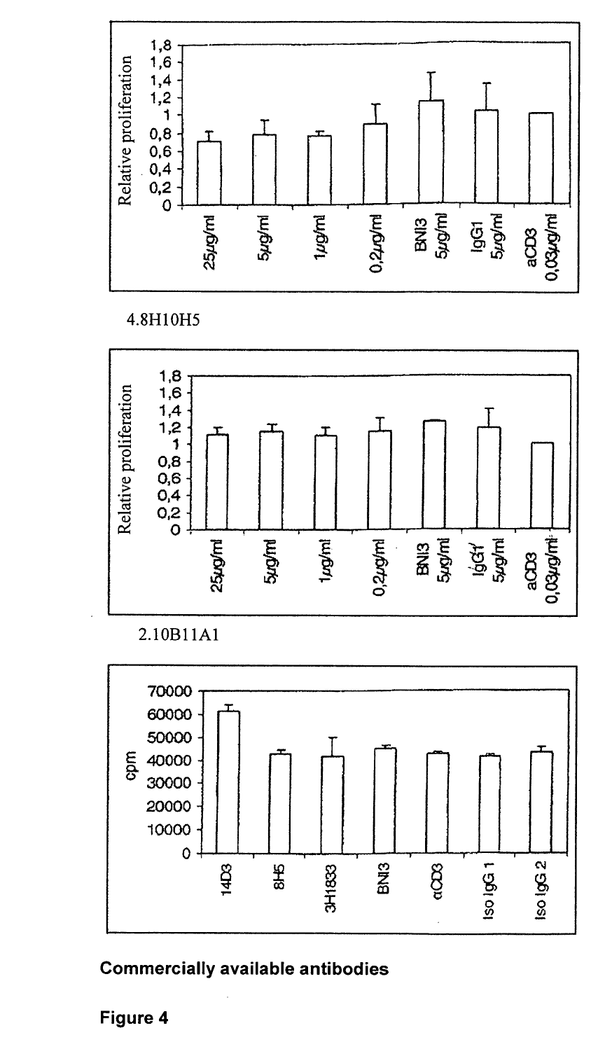

FIG. 4 shows the inhibiting effect of the anti-CTLA-4 antibody 4.8H10H5 on the proliferation of human PBMCs. The objective of this proliferation inhibition assay was to identify an antibody with a new type of function, compared to the already known CTLA-4 specific antibodies. An important characteristic of a superagonistic antibody was defined to be the ability to reduce the proliferation of human PBMC. Another criterion was that this effect can be observed with soluble, not artificially interlinked antibody. Those antibodies were evaluated positively, which reduced an anti-CD3 (or superagonistic anti-CD28; not shown) induced proliferation of the T cells by at least 25%. Readout system was the measuring of the proliferation with ³H thymidine incorporation. In this assay system, the CTLA-4 specific antibodies were administered at the same time as the activating anti-CD3 antibody and the proliferation was determined after 63-66 hours. Based on the above criteria, antibody 4.8H10H5 was able to inhibit the proliferation of T cells. For comparison, an antibody is mentioned, which in this assay is not positively evaluated (2.10B11A1). Shown is the relative proliferation compared to the positive control (anti-CD3-induced proliferation). For the purpose of further controls, the respective isotype control (IgG1 or IgG2) and a commercially available antibody (BNI3, BD Pharmingen) were also carried. The carried commercial antibodies (14D3, 8H5, 3H1833, BNI3) with specificity for CTLA-4 remained without effect.

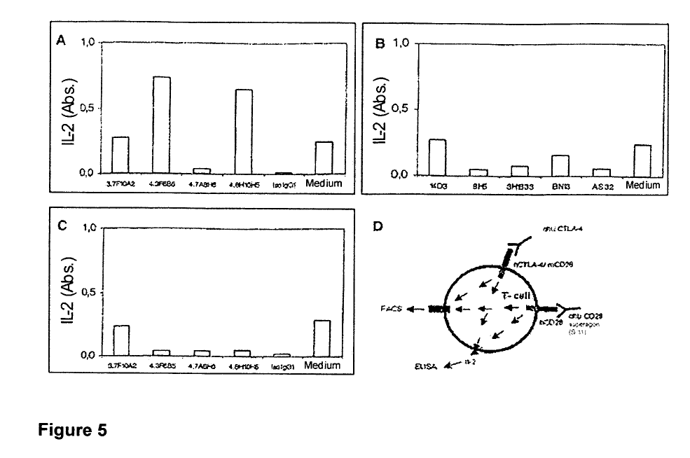

FIG. 5 shows the stimulating effect of the anti-CTLA-4 antibodies 4.8H10H5 and 4.3F6B5 on the IL-2 production of Jurkat E6.1 cells that express a chimaerous CTLA-4/CD28 molecule. For this cell-autonomous readout system for the functional characterisation of the CTLA-4-specific MAK, Jurkat E6.1 cells were used that express a chimaerous CTLA-4/CD28 receptor on their surface (D). This consists of the extracellular domain of the CTLA-4 receptor and the transmembrane and intercellular domain of CD28. The activation of the chimaerous receptor by CTLA-4-specific MAK induces CD28-specific activation markers, such as IL-2 or CD69, which can be measured with ELISA or FACS analysis. In this system, potentially superagonistic CTLA-4-specific antibodies can be identified with the help of CD28-specific activation markers. Control antibodies and CTLA-4-specific antibodies were cross-linked (using sheep anti mouse Ig) and incubated with 1*10⁵ transfixed Jurkat E6.1 cells for 48 hours. As activation marker, the IL-2 production was measured with ELISA.

As controls, isotype controls and commercially available antibodies with specificity for CTLA-4 were also carried. (A) Effect of the CTLA-4-specific antibodies (1 μg/ml) on the IL-2 production of transfixed Jurkat cells that express a chimaerous CTLA-4/CD28 receptor. (B) Effect of the commercially available CTLA-4-specific antibodies (1 μg/ml) on the IL-2 production of transfixed Jurkat cells that express a chimaerous CTLA-4/CD28 receptor. (C) Effect of the CTLA-4-specific antibodies (1 μg/ml) on the IL-2 production of not-transfixed Jurkat E6.1 cells that are missing the chimaerous receptor.

(Representative experiments). Two of the tested antibodies (4.3F6B5, 4.8H10H5) induce the IL-2 production of the transfixed Jurkat cells through activation of the chimaerous receptor whilst none of the commercially available antibodies with CTLA-4 specificity were able to do that.

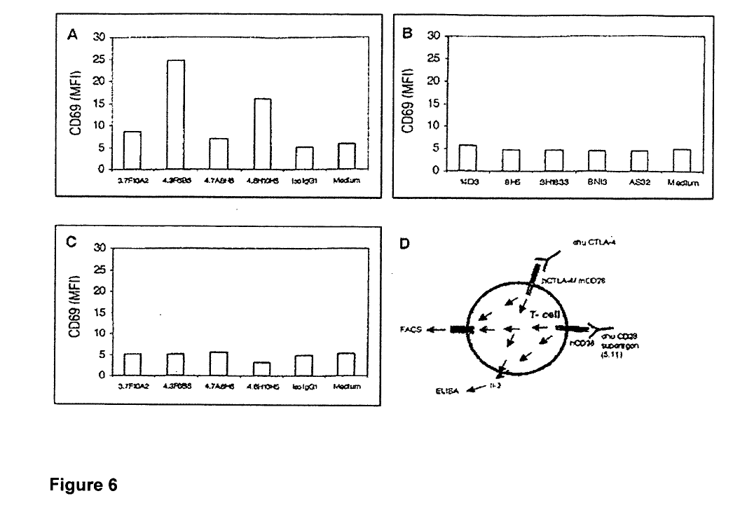

FIG. 6 shows the stimulating effect of the anti-CTLA-4 antibodies 4.8H10H5 and 4.3F6B5 on the CD69 induction of Jurkat E6.1 cells that express a chimaerous CTLA-4/CD28 molecule. (D) Using the assay system described in FIG. 6, the CD69 expression was measured as further activation marker in addition to the IL-2 production, using the FACS analysis. In contrast to the IL-2 production, CD69 is an early activation marker and can be detected as early as 4 hours of incubation of the antibodies with the transfixed cells. (A) Effect of the CTLA-4-specific antibodies (1 μg/ml) on the CD69 expression of transfixed Jurkat cells that express a chimaerous CTLA-4/CD28 receptor. (B) Effect of the commercially available CTLA-4-specific antibodies (1 μg/ml) on the CD69 expression of transfixed Jurkat cells that express a chimaerous CTLA-4/CD28 receptor. (C) Effect of the CTLA-4-specific antibodies (1 μg/ml) on the CD69 expression of not-transfixed Jurkat E6.1 cells that are missing the chimaerous receptor.

(Representative experiments). As was shown for IL-2, the antibodies 4.3F6B5 and 4.8H10H5 are able to activate the chimaerous receptor, to trigger a signal transduction and to induce the CD69 expression. None of the commercially available antibodies with CTLA-4 specificity were able to do that. (Representative experiments).

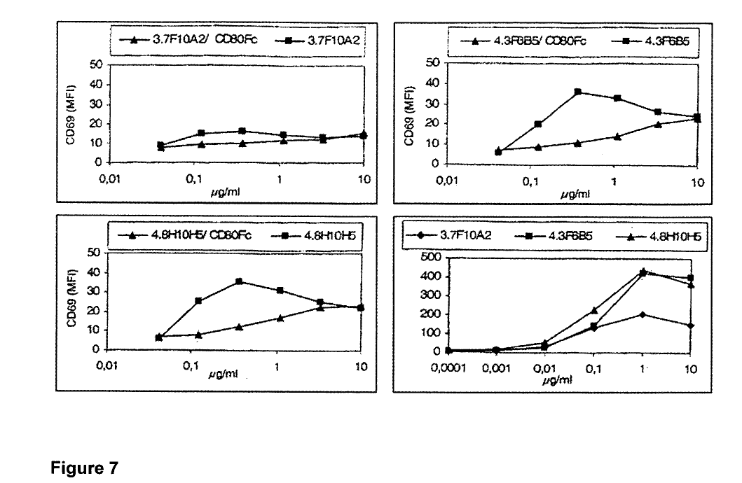

FIG. 7 shows that the stimulating effect of the anti-CTLA-4 antibodies 4.8H10H5 and 4.3F6B5 on the chimaerous CTLA-4/CD28 construct could be reduced by CD80 Fc protein. Jurkat E6.1 cells expressing the chimaerous CTLA-4/CD28 receptor (see FIGS. 5, 6) were incubated with increasing concentrations of antibodies and each time 1 μg/ml CD80 Fc protein. The antibody-induced CD69 expression is then reduced by CD80Fc as soon as the recombinant protein is incubated in excess compared to the antibody. Rectangular squares show the respective CD69 induction without CD80 co-incubation, the curves represented by triangles show the CD69 induction by the respective antibodies reduced by 1 μg/ml CD80 Fc. In addition, the figure shows the concentration-related binding of the antibodies to the Jurkat cells.

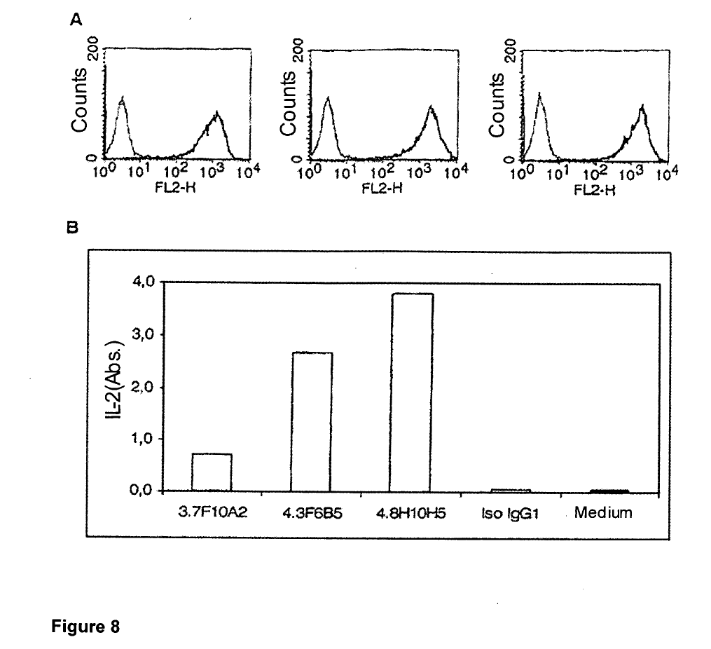

FIG. 8 shows the crossreactivity of the anti-CTLA-4 antibodies 4.8H10H5 and 4.3F6B5 with the CTLA-4 molecule of the rat and the stimulating functionality of the antibodies in a chimaerous receptor assay.

(A) The binding of anti-CTLA-4 antibodies to rat CTLA-4 was demonstrated with BW cells expressing the extracellular domain of the rat receptor on their surface. Analogous to the chimaerous receptor on Jurkat cells (FIG. 5), these cells express a chimaerous CTLA-4 CD28 receptor consisting of rat CTLA-4 (extracellular domain) and mouse CD28 (transmembrane/intracellular domain) (light line: anti-CTLA-4 antibodies, dark curve: isotype control). (B) For the cross-reactive antibodies 4.3F6B5 and 4.8H10H5 it was possible to demonstrate the activation of the chimaerous receptor using the IL-2 induction. As in the assay system described in FIG. 5, it is possible to use transfixed BW cells to identify superagonistic CTLA-4-specific antibodies using CD28-specific activation markers (IL-2). In a control experiment, the antibodies were not able to induce an IL-2 production on BW cells without the chimaerous receptor.

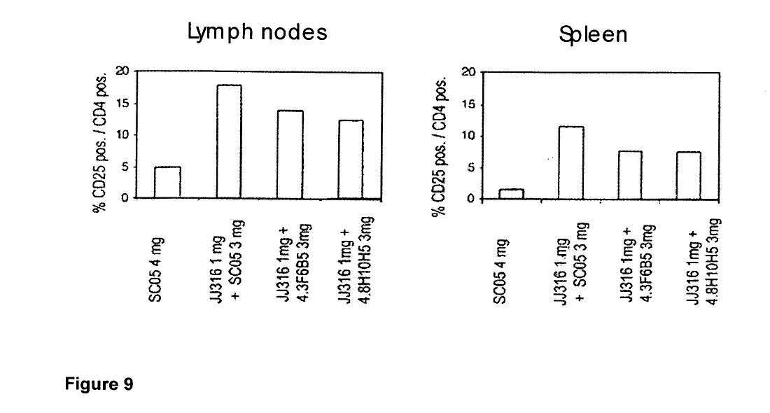

FIG. 9 shows the inhibiting in vivo effect of the anti-CTLA-4 antibodies 4.8H10H5 and 4.3F6B5 on the CD28 superMAB-induced activation of T cells in the rat. For this purpose, activating superagonistic rat-specific CD28 antibodies (JJ316) were applied i.v. together with CTLA-4-specific antibodies/isotype control rats. After three days cell suspensions were obtained from lymph nodes and spleen and analysed for the activation marker CD25 using the FACS method. Overall, three experiments were carried out with varying antibody concentrations. Both CTLA-4-specific antibodies tested reduced the JJ316-induced CD25 expression on lymph node as well as spleen cells in 3 independent experiments by approximately 30-40%. A representative result is shown.

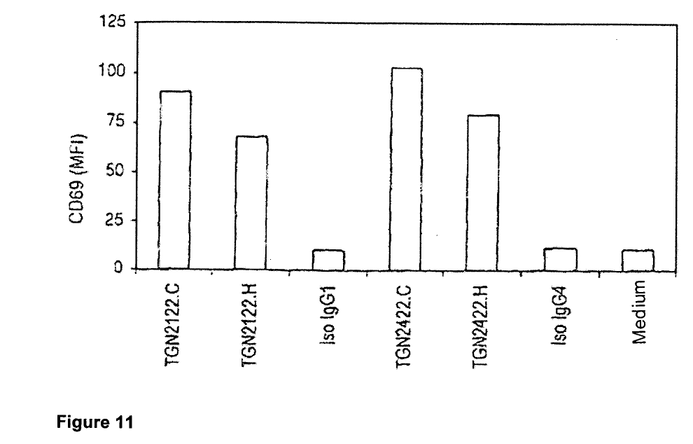

FIG. 11 illustrates the stimulating effect of the humanised anti-CTLA-4 antibodies (1 μg/ml) in vitro on the CD69 expression of Jurkat E6.1 cells expressing a chimaerous CTLA-4/CD28 receptor. The same process as described in FIG. 6 was adopted. One can see from FIG. 11 that all humanised antibodies of the invention, both of the isotype IgG1 and IgG4, induce effectively the CD69 surface expression whilst the isotype control/the addition of cell culture medium remained without effect (representative result). This demonstrates that the functionally new properties of antibodies 4.3F6B5 and 4.8H10H5 have remained intact during the humanisation process.

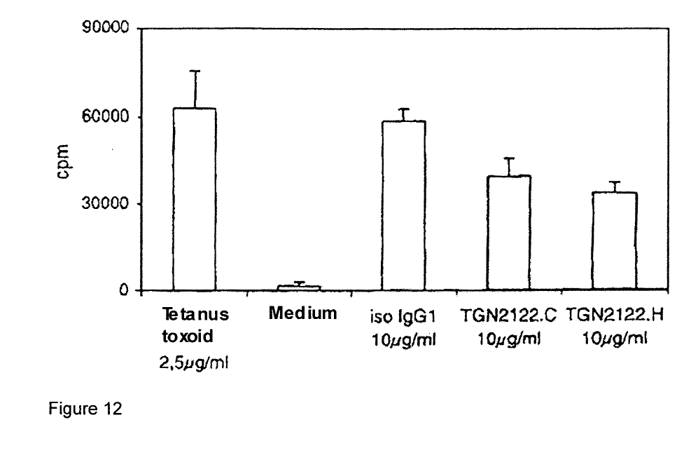

FIG. 12 illustrates the inhibiting effect of antibodies TGN2122.C and TGN2122.H on the proliferation of ex vivo stimulated human PBMCs. In a recall response assay, 10̂5 human PBMCs of healthy donors were activated with 2.5 μg/ml tetanus toxoid and simultaneously the corresponding CTLA-4-specific antibody/the isotype control was added to the assay preparation. The proliferation was measured by ³H thymidine incorporation after an incubation of 120 hours. ³H thymidine was added to the assay preparation for the last 15-18 hours of the test and the ³H thymidine incorporation determined. As controls, the respective isotype control and a preparation containing non-activated cells without antibodies were also carried out. Both antibodies were able to effectively inhibit the tetanus toxoid induced proliferation. By contrast, the isotype control did not show this inhibiting effect. These tests demonstrate the superagonistic properties of the antibodies that are the subject of the invention, since the inhibition occurred in soluble form, i.e. without artificial cross-linking.

Where Xₙ is included in sequences, the ‘n’ may vary by ±1.

TABLE 1
Bond characteristics of new anti-CTLA-4 antibodies

| | Bond to | | | | | |
|---|---|---|---|---|---|---|
| | | | HumanCTLA-4 | | | |
| | Human | Human C″D | on | Human | Human | Rat |
| | CTLA-4 on | loop on | activated | CD28 on | ICOS on | CTLA-4 on |
| Clone | transfectants | transfectants | PBMCs | transfectants | transfectants | transfectants |
| 3.7F10A2 | + | + | + | − | − | + |
| 4.3F6B5 | + | − | + | − | − | + |
| 4.4A7F4 | + | − | + | − | − | + |
| 4.6C1E3 | + | − | + | − | − | − |
| 4.7A8H6 | + | + | + | − | − | − |
| 4.7E11F1 | + | − | + | − | − | − |
| 4.8H10H5 | + | − | + | − | − | + |

TABLE 2
4.3F6B5
Heavy chain

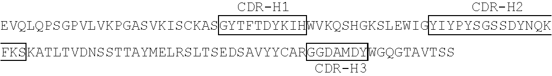

Light chain

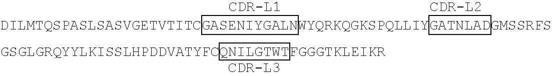

4.8H10H5
Heavy chain

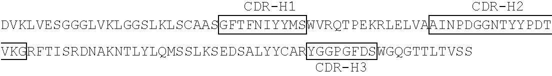

Light chain

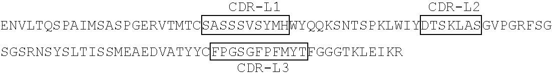

TABLE 3
TGN2122.H HC Amino acid sequence

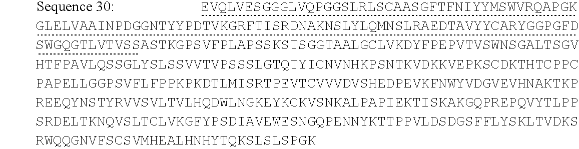

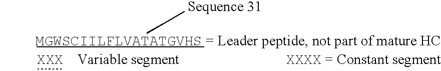

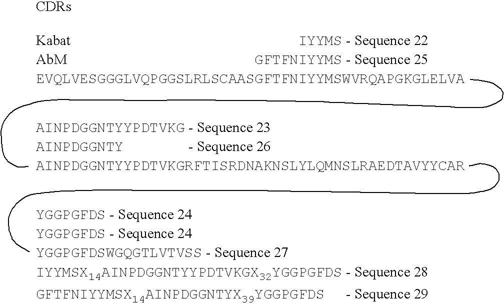

TABLE 4

TGN2122.H HC Nucleotide sequence

(Sequence 40)

| | |
|---|---|
| 1 | ATGGGATGGA GCTGTATCAT CCTCTTCTTG GTAGCAACAG CTACAGGTAA |
| 51 | GGGGCTCACA GTAGCAGGCT TGAGGTCTGG ACATATATAT GGGTGACAAT |
| 101 | GACATCCACT TTGCCTTTCT CTCCACAGGT GTGCATTCCG AGGTGCAGCT |
| 151 | GGTGGAGAGC GGCGGCGGCC TGGTGCAGCC CGGCGGCAGC CTGAGGCTGA |
| 201 | GCTGCGCCGC CAGCGGCTTC ACCTTCAACA TCTACTACAT GAGCTGGGTG |
| 251 | AGGCAGGCCC CCGGCAAGGG CCTGGAGCTG GTGGCCGCCA TCAACCCCGA |
| 301 | CGGCGGCAAC ACCTACTACC CCGACACCGT GAAGGGCAGG TTCACCATCA |
| 351 | GCAGGGACAA CGCCAAGAAC AGCCTGTACC TGCAGATGAA CAGCCTGAGG |
| 401 | GCCGAGGACA CCGCCGTGTA CTACTGCGCC AGGTACGGCG GCCCCGGCTT |
| 451 | CGACAGCTTG GGCCAGGGCA CCCTGGTGAC CGTGAGCAGC GGTGAGTCGT |
| 501 | ACGCTAGCAA GCTTTCTGGG GCAGGCCAGG CCTGACCTTG GCTTTGGGGC |
| 551 | AGGGAGGGGG CTAAGGTGAG GCAGGTGGCG CCAGCCAGGT GCACACCCAA |
| 601 | TGCCCATGAG CCCAGACACT GGACGCTGAA CCTCGCGGAC AGTTAAGAAC |
| 651 | CCAGGGGCCT CTGCGCCCTG GGCCCAGCTC TGTCCCACAC CGCGGTCACA |
| 701 | TGGCACCCTC CTCCAAGAGC ACCTCTGGGG GCACAGCGGC CCTGGGCTGC |
| 751 | TGGCACCCTC CTCCAAGAGC ACCTCTGGGG GCACAGCGGC CCTGGGCTGC |
| 801 | CTGGTCAAGG ACTACTTCCC CGAACCGGTG ACGGTGTCGT GGAACTCAGG |
| 851 | CGCCCTGACC AGCGGCGTGC ACACCTTCCC GGCTGTCCTA CAGTCCTCAG |
| 901 | GACTCTACTC CCTCAGCAGC GTGGTGACCG TGCCCTCCAG CAGCTTGGGC |
| 951 | ACCCAGACCT ACATCTGCAA CGTGAATCAC AAGCCCAGCA ACACCAAGGT |
| 1001 | GGACAAGAAA GTTGGTGAGA GGCCAGCACA GGGAGGGAGG GTGTCTGCTG |
| 1051 | GAAGCCAGGC TCAGCGCTCC TGCCTGGACG CATCCCGGCT ATGCAGCCCC |
| 1101 | AGTCCAGGGC AGCAAGGCAG GCCCCGTCTG CCTCTTCACC CGGAGGCCTC |
| 1151 | TGCCCGCCCC ACTCATGCTC AGGGAGAGGG TCTTCTGGCT TTTTCCCAGG |
| 1201 | CTCTGGGCAG GCACAGGCTA GGTGCCCCTA ACCCAGGCCC TGCACACAAA |
| 1251 | GGGGCAGGTG CTGGGCTCAG ACCTGCCAAG AGCCATATCC GGGAGGACCC |
| 1301 | TGCCCCTGAC CTAAGCCCAC CCCAAAGGCC AAACTCTCCA CTCCCTCAGC |
| 1351 | TCGGACACCT TCTCTCCTCC CAGATTCCAG TAACTCCCAA TCTTCTCTCT |
| 1401 | GCAGAGCCCA AATCTTGTGA CAAAACTCAC ACATGCCCAC CGTGCCCAGG |
| 1451 | TAAGCCAGCC CAGGCCTCGC CCTCCAGCTC AAGGCGGGAC AGGTGCCCTA |
| 1501 | GAGTAGCCTG CATCCAGGGA CAGGCCCCAG CCGGGTGCTG ACACGTCCAC |
| 1551 | CTCCATCTCT TCCTCAGCAC CTGAACTCCT GGGGGGACCG TCAGTCTTCC |
| 1601 | TCTTCCCCCC AAAACCCAAG GACACCCTCA TGATCTCCCG GACCCCTGAG |
| 1651 | GTCACATGCG TGGTGGTGGA CGTGAGCCAC GAAGACCCTG AGGTCAAGTT |
| 1701 | CAACTGGTAC GTGGACGGCG TGGAGGTGCA TAATGCCAAG ACAAAGCCGC |
| 1751 | GGGAGGAGCA GTACAACAGC ACGTACCGGG TGGTCAGCGT CCTCACCGTC |
| 1801 | CTGCACCAGG ACTGGCTGAA TGGCAAGGAG TACAAGTGCA AGGTCTCCAA |
| 1851 | CAAAGCCCTC CCAGCCCCCA TCGAGAAAAC CATCTCCAAA GCCAAAGGTG |
| 1901 | GGACCCGTGG GGTGCGAGGG CCACATGGAC AGAGGCCGGC TCGGCCCACC |
| 1951 | CTCTGCCCTG AGAGTGACCG CTGTACCAAC CTCTGTCCCT ACAGGGCAGC |
| 2001 | CCCGAGAACC ACAGGTGTAC ACCCTGCCCC CATCCCGGGA TGAGCTGACC |
| 2051 | AAGAACCAGG TCAGCCTGAC CTGCCTGGTC AAAGGCTTCT ATCCCAGCGA |
| 2101 | CATCGCCGTG GAGTGGGAGA GCAATGGGCA GCCGGAGAAC AACTACAAGA |
| 2151 | CCACGCCTCC CGTGCTGGAC TCCGACGGCT CCTTCTTCCT CTACAGCAAG |
| 2201 | CTCACCGTGG ACAAGAGCAG GTGGCAGCAG GGGAACGTCT TCTCATGCTC |
| 2251 | CGTGATGCAT GAGGCTCTGC ACAACCACTA CACGCAGAAG AGCCTCTCCC |
| 2301 | TGTCTCCGGG TAAATGA |

TABLE 5
TGN2422.H HC Amino acid sequence

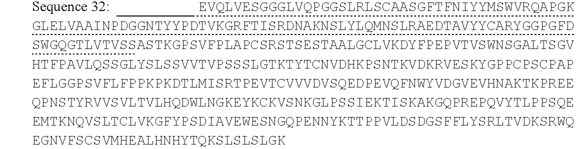

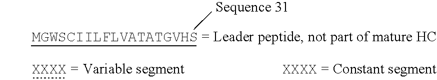

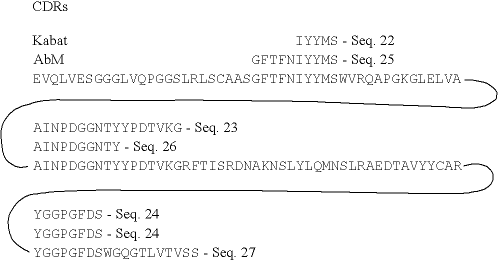

TABLE 6

TGN2422.H HC Nucleotide sequence

(Sequence 41)

| | |
|---|---|
| 1 | ATGGGATGGA GCTGTATCAT CCTCTTCTTG GTAGCAACAG CTACAGGTAA |
| 51 | GGGGCTCACA GTAGCAGGCT TGAGGTCTGG ACATATATAT GGGTGACAAT |
| 101 | GACATCCACT TTGCCTTTCT CTCCACAGGT GTGCATTCCG AGGTGCAGCT |
| 151 | GGTGGAGAGC GGCGGCGGCC TGGTGCAGCC CGGCGGCAGC CTGAGGCTGA |
| 201 | GCTGCGCCGC CAGCGGCTTC ACCTTCAACA TCTACTACAT GAGCTGGGTG |
| 251 | AGGCAGGCCC CCGGCAAGGG CCTGGAGCTG GTGGCCGCCA TCAACCCCGA |
| 301 | CGGCGGCAAC ACCTACTACC CCGACACCGT GAAGGGCAGG TTCACCATCA |
| 351 | GCAGGGACAA CGCCAAGAAC AGCCTGTACC TGCAGATGAA CAGCCTGAGG |
| 401 | GCCGAGGACA CCGCCGTGTA CTACTGCGCC AGGTACGGCG GCCCCGGCTT |
| 451 | CGACAGCTTG GGCCAGGGCA CCCTGGTGAC CGTGAGCAGC GGTGAGTCGT |
| 501 | ACGCTAGCAA GCTTTCTGGG GCAGGCCGGG CCTGACTTTG GCTGGGGGCA |
| 551 | GGGAGGGGGC TAAGGTGACG CAGGTGGCGC CAGCCAGGTG CACACCCAAT |
| 601 | GCCCATGAGC CCAGACACTG GACCCTGCAT GGACCATCGC GGATAGACAA |
| 651 | GAACCGAGGG GCCTCTGCGC CCTGGGCCCA GCTGTGTCCC ACACCGCGGT |
| 701 | CACATGGCAC CACCTCTCTT GCAGCTTCCA CCAAGGGCCC ATCCGTCTTC |
| 751 | CCCCTGGCGC CCTGCTCCAG GAGCACCTCC GAGAGCACAG CCGCCCTGGG |
| 801 | CTGCCTGGTC AAGGACTACT TCCCCGAACC GGTGACGGTG TCGTGGAACT |
| 851 | CAGGCGCCCT GACCAGCGGC GTGCACACCT TCCCGGCTGT CCTACAGTCC |
| 901 | TCAGGACTCT ACTCCCTCAG CAGCGTGGTG ACCGTGCCCT CCAGCAGCTT |
| 951 | GGGCACGAAG ACCTACACCT GCAACGTAGA TCACAAGCCC AGCAACACCA |
| 1001 | AGGTGGACAA GAGAGTTGGT GAGAGGCCAG CACAGGGAGG GAGGGTGTCT |
| 1051 | GCTGGAAGCC AGGCTCAGCC CTCCTGCCTG GACGCACCCC GGCTGTGCAG |
| 1101 | CCCCAGCCCA GGGCAGCAAG GCATGCCCCA TCTGTCTCCT CACCCGGAGG |
| 1151 | CCTCTGACCA CCCCACTCAT GCTCAGGGAG AGGGTCTTCT GGATTTTTCC |
| 1201 | ACCAGGCTCC GGGCAGCCAC AGGCTGGATG CCCCTACCCC AGGCCCTGCG |
| 1251 | CATACAGGGG CAGGTGCTGC GCTCAGACCT GCCAAGAGCC ATATCCGGGA |
| 1301 | GGACCCTGCC CCTGACCTAA GCCCACCCCA AAGGCCAAAC TCTCCACTCC |
| 1351 | CTCAGCTCAG ACACCTTCTC TCCTCCCAGA TCTGAGTAAC TCCCAATCTT |
| 1401 | CTCTCTGCAG AGTCCAAATA TGGTCCCCCA TGCCCATCAT GCCCAGGTAA |
| 1451 | GCCAACCCAG GCCTCGCCCT CCAGCTCAAG GCGGGACAGG TGCCCTAGAG |
| 1501 | TAGCCTGCAT CCAGGGACAG GCCCCAGCCG GGTGCTGACG CATCCACCTC |
| 1551 | CATCTCTTCC TCAGCACCTG AGTTCCTGGG GGGACCATCA GTCTTCCTGT |
| 1601 | TCCCCCCAAA ACCCAAGGAC ACTCTCATGA TCTCCCGGAC CCCTGAGGTC |
| 1651 | ACGTGCGTGG TGGTGGACGT GAGCCAGGAA GACCCCGAGG TCCAGTTCAA |
| 1701 | CTGGTACGTG GATGGCGTGG AGGTGCATAA TGCCAAGACA AAGCCGCGGG |
| 1751 | AGGAGCAGTT CAACAGCACG TACCGTGTGG TCAGCGTCCT CACCGTCCTG |
| 1801 | CACCAGGACT GGCTGAACGG CAAGGAGTAC AAGTGCAAGG TCTCCAACAA |
| 1851 | AGGCCTCCCG TCCTCCATCG AGAAAACCAT CTCCAAAGCC AAAGGTGGGA |
| 1901 | CCCACGGGGT GCGAGGGCCA CATGGACAGA GGTCAGCTCG GCCCACCCTC |
| 1951 | TGCCCTGGGA GTGACCGCTG TGCCAACCTC TGTCCCTACA GGGCAGCCCC |
| 2001 | GAGAGCCACA GGTGTACACC CTGCCCCCAT CCCAGGAGGA GATGACCAAG |
| 2051 | AACCAGGTCA GCCTGACCTG CCTGGTCAAA GGCTTCTACC CCAGCGACAT |
| 2101 | CGCCGTGGAG TGGGAGAGCA ATGGGCAGCC GGAGAACAAC TACAAGACCA |
| 2151 | CGCCTCCCGT GCTGGACTCC GACGGCTCCT TCTTCCTCTA CAGCAGGCTA |
| 2201 | ACCGTGGACA AGAGCAGGTG GCAGGAGGGG AATGTCTTCT CATGCTCCGT |
| 2251 | GATGCATGAG GCTCTGCACA ACCACTACAC ACAGAAGAGC CTCTCCCTGT |
| 2301 | CTCTGGGTAA ATGA |

TABLE 7
TGN2122/TGN2422.H-kappa LC Amino acid sequence

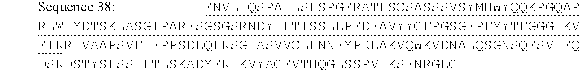

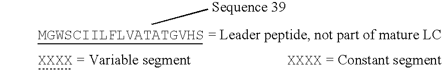

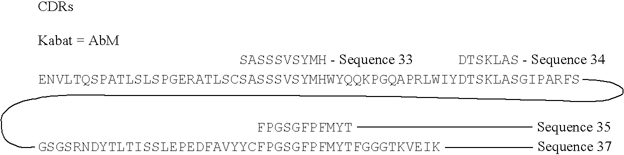

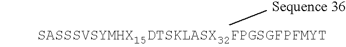

TABLE 8

TGN2122/TGN2422.H-kappa LC Nucleotide sequence

(Sequence 42)

| | |
|---|---|
| 1 | ATGGGATGGA GCTGTATCAT CCTCTTCTTG GTAGCAACAG CTACAGGTAA |
| 51 | GGGGCTCACA GTAGCAGGCT TGAGGTCTGG ACATATATAT GGGTGACAAT |
| 101 | GACATCCACT TTGCCTTTCT CTCCACAGGT GTGCATTCCG AGAACGTGCT |
| 151 | GACCCAGAGC CCCGCCACCC TGAGCCTGAG CCCCGGCGAG AGGGCCACCC |
| 201 | TGAGCTGCAG CGCCAGCAGC AGCGTGAGCT ACATGCACTG GTACCAGCAG |
| 251 | AAGCCCGGCC AGGCCCCCAG GCTGTGGATC TACGACACCA GCAAGCTGGC |
| 301 | CAGCGGCATC CCCGCCAGGT TCAGCGGCAG CGGCAGCAGG AACGACTACA |
| 351 | CCCTGACCAT CAGCAGCCTG GAGCCCGAGG ACTTCGCCGT GTACTACTGC |
| 401 | TTCCCCGGCA GCGGCTTCCC CTTCATGTAC ACCTTCGGCG GCGGCACCAA |
| 451 | GGTGGAGATC AAGCGTGAGT CGTACGCTAG CAAGCTTGAT ATCGAATTCT |
| 501 | AAACTCTGAG GGGGTCGGAT GACGTGGCCA TTCTTTGCCT AAAGCATTGA |
| 551 | GTTTACTGCA AGGTCAGAAA AGCATGCAAA GCCCTCAGAA TGGCTGCAAA |
| 601 | GAGCTCCAAC AAAACAATTT AGAACTTTAT TAAGGAATAG GGGGAAGCTA |
| 651 | GGAAGAAACT CAAAACATCA AGATTTTAAA TACGCTTCTT GGTCTCCTTG |
| 701 | CTATAATTAT CTGGGATAAG CATGCTGTTT TCTGTCTGTC CCTAACATGC |
| 751 | CCTGTGATTA TCCGCAAACA ACACACCCAA GGGCAGAACT TTGTTACTTA |
| 801 | AACACCATCC TGTTTGCTTC TTTCCTCAGG AACTGTGGCT GCACCATCTG |
| 851 | TCTTCATCTT CCCGCCATCT GATGAGCAGT TGAAATCTGG AACTGCCTCT |
| 901 | GTTGTGTGCC TGCTGAATAA CTTCTATCCC AGAGAGGCCA AAGTACAGTG |
| 951 | GAAGGTGGAT AACGCCCTCC AATCGGGTAA CTCCCAGGAG AGTGTCACAG |
| 1001 | AGCAGGACAG CAAGGACAGC ACCTACAGCC TCAGCAGCAC CCTGACGCTG |
| 1051 | AGCAAAGCAG ACTACGAGAA ACACAAAGTC TACGCCTGCT AAGTCACCCA |
| 1101 | TCAGGGCCTG AGCTCGCCCG TCACAAAGAG CTTCAACAGG GGAGAGTGTT |
| 1151 | AG |

TABLE 9
TGN2122.C HC Amino acid sequence

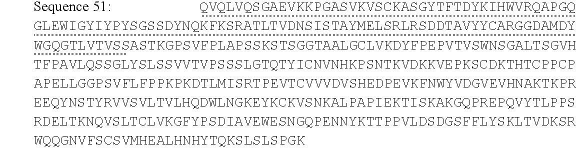

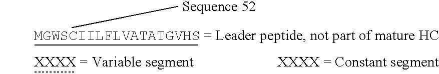

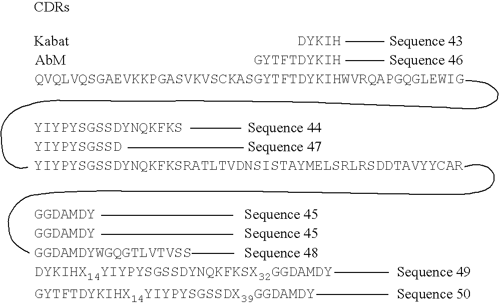

TABLE 10

TGN2122.C HC Nucleotide sequence

(Sequence 61)

| | |
|---|---|
| 1 | ATGGGATGGA GCTGTATCAT CCTCTTCTTG GTAGCAACAG CTACAGGTAA |
| 51 | GGGGCTCACA GTAGCAGGCT TGAGGTCTGG ACATATATAT GGGTGACAAT |
| 101 | GACATCCACT TTGCCTTTCT CTCCACAGGT GTGCATTCCC AGGTGCAGCT |
| 151 | GGTGCAGAGC GGCGCCGAGG TGAAGAAGCC CGGCGCCAGC GTGAAGGTGA |
| 201 | GCTGCAAGGC CAGCGGCTAC ACCTTCACCG ACTACAAGAT CCACTGGGTG |
| 251 | AGGCAGGCCC CCGGCCAGGG CCTGGAGTGG ATCGGCTACA TCTACCCCTA |
| 301 | CAGCGGCAGC AGCGACTACA ACCAGAAGTT CAAGAGCAGG GCCACCCTGA |
| 351 | CCGTGGACAA CAGCATCAGC ACCGCCTACA TGGAGCTGAG CAGGCTGAGG |
| 401 | AGCGACGACA CCGCCGTGTA CTACTGCGCC AGGGGCGGCG ACGCCATGGA |
| 451 | CTACTGGGGC CAGGGCACCC TGGTGACCGT GAGCAGCGGT GAGTCGTACG |
| 501 | CTAGCAAGCT TTCTGGGGCA GGCCAGGCCT GACCTTGGCT TTGGGGCAGG |
| 551 | GAGGGGGCTA AGGTGAGGCA GGTGGCGCCA GCCAGGTGCA CACCCAATGC |
| 601 | CCATGAGCCC AGACACTGGA CGCTGAACCT CGCGGACAGT TAAGAACCCA |
| 651 | GGGGCCTCTG CGCCCTGGGC CCAGCTCTGT CCCACACCGC GGTCACATGG |
| 701 | CACCACCTCT CTTGCAGCCT CCACCAAGGG CCCATCGGTC TTCCCCCTGG |
| 751 | CACCCTCCTC CAAGAGCACC TCTGGGGGCA CAGCGGCCCT GGGCTGCCTG |
| 801 | GTCAAGGACT ACTTCCCCGA ACCGGTGACG GTGTCGTGGA ACTCAGGCGC |
| 851 | CCTGACCAGC GGCGTGCACA CCTTCCCGGC TGTCCTACAG TCCTCAGGAC |
| 901 | TCTACTCCCT CAGCAGCGTG GTGACCGTGC CCTCCAGCAG CTTGGGCACC |
| 951 | CAGACCTACA TCTGCAACGT GAATCACAAG CCCAGCAACA CCAAGGTGGA |
| 1001 | CAAGAAAGTT GGTGAGAGGC CAGCACAGGG AGGGAGGGTG TCTGCTGGAA |
| 1051 | GCCAGGCTCA GCGCTCCTGC CTGGACGCAT CCCGGCTATG CAGCCCCAGT |
| 1101 | CCAGGGCAGC AAGGCAGGCC CCGTCTGCCT CTTCACCCGG AGGCCTCTGC |
| 1151 | CCGCCCCACT CATGCTCAGG GAGAGGGTCT TCTGGCTTTT TCCCAGGCTC |
| 1201 | TGGGCAGGCA CAGGCTAGGT GCCCCTAACC CAGGCCCTGC ACACAAAGGG |
| 1251 | GCAGGTGCTG GGCTCAGACC TGCCAAGAGC CATATCCGGG AGGACCCTGC |
| 1301 | CCCTGACCTA AGCCCACCCC AAAGGCCAAA CTCTCCACTC CCTCAGCTCG |
| 1351 | GACACCTTCT CTCCTCCCAG ATTCCAGTAA CTCCCAATCT TCTCTCTGCA |
| 1401 | GAGCCCAAAT CTTGTGACAA AACTCACACA TGCCCACCGT GCCCAGGTAA |
| 1451 | GCCAGCCCAG GCCTCGCCCT CCAGCTCAAG GCGGGACAGG TGCCCTAGAG |
| 1501 | TAGCCTGCAT CCAGGGACAG GCCCCAGCCG GGTGCTGACA CGTCCACCTC |
| 1551 | CATCTCTTCC TCAGCACCTG AACTCCTGGG GGGACCGTCA GTCTTCCTCT |
| 1601 | TCCCCCCAAA ACCCAAGGAC ACCCTCATGA TCTCCCGGAC CCCTGAGGTC |
| 1651 | ACATGCGTGG TGGTGGACGT GAGCCACGAA GACCCTGAGG TCAAGTTCAA |
| 1701 | CTGGTACGTG GACGGCGTGG AGGTGCATAA TGCCAAGACA AAGCCGCGGG |
| 1751 | AGGAGCAGTA CAACAGCACG TACCGGGTGG TCAGCGTCCT CACCGTCCTG |
| 1801 | CACCAGGACT GGCTGAATGG CAAGGAGTAC AAGTGCAAGG TCTCCAACAA |
| 1851 | AGCCCTCCCA GCCCCCATCG AGAAAACCAT CTCCAAAGCC AAAGGTGGGA |
| 1901 | CCCGTGGGGT GCGAGGGCCA CATGGACAGA GGCCGGCTCG GCCCACCCTC |
| 1951 | TGCCCTGAGA GTGACCGCTG TACCAACCTC TGTCCCTACA GGGCAGCCCC |
| 2001 | GAGAACCACA GGTGTACACC CTGCCCCCAT CCCGGGATGA GCTGACCAAG |
| 2051 | AACCAGGTCA GCCTGACCTG CCTGGTCAAA GGCTTCTATC CCAGCGACAT |
| 2101 | CGCCGTGGAG TGGGAGAGCA ATGGGCAGCC GGAGAACAAC TACAAGACCA |
| 2151 | CGCCTCCCGT GCTGGACTCC GACGGCTCCT TCTTCCTCTA CAGCAAGCTC |
| 2201 | ACCGTGGACA AGAGCAGGTG GCAGCAGGGG AACGTCTTCT CATGCTCCGT |
| 2251 | GATGCATGAG GCTCTGCACA ACCACTACAC GCAGAAGAGC CTCTCCCTGT |
| 2301 | CTCCGGGTAA ATGA |

TABLE 11
TGN2422.C HC Amino acid sequence

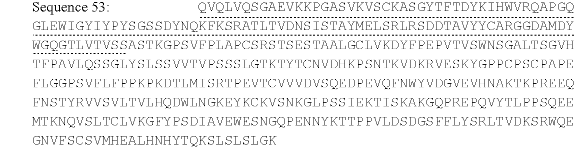

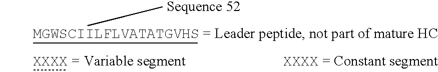

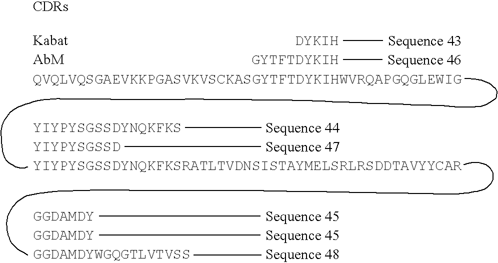

TABLE 12

TGN2122.C HC Nukleotide sequence

(Sequence 62)

| | |
|---|---|
| 1 | ATGGGATGGA GCTGTATCAT CCTCTTCTTG GTAGCAACAG CTACAGGTAA |
| 51 | GGGGCTCACA GTAGCAGGCT TGAGGTCTGG ACATATATAT GGGTGACAAT |
| 101 | GACATCCACT TTGCCTTTCT CTCCACAGGT GTGCATTCCC AGGTGCAGCT |
| 151 | GGTGCAGAGC GGCGCCGAGG TGAAGAAGCC CGGCGCCAGC GTGAAGGTGA |
| 201 | GCTGCAAGGC CAGCGGCTAC ACCTTCACCG ACTACAAGAT CCACTGGGTG |
| 251 | AGGCAGGCCC CCGGCCAGGG CCTGGAGTGG ATCGGCTACA TCTACCCCTA |
| 301 | CAGCGGCAGC AGCGACTACA ACCAGAAGTT CAAGAGCAGG GCCACCCTGA |
| 351 | CCGTGGACAA CAGCATCAGC ACCGCCTACA TGGAGCTGAG CAGGCTGAGG |
| 401 | AGCGACGACA CCGCCGTGTA CTACTGCGCC AGGGGCGGCG ACGCCATGGA |
| 451 | CTACTGGGGC CAGGGCACCC TGGTGACCGT GAGCAGCGGT GAGTCGTACG |
| 501 | CTAGCAAGCT TTCTGGGGCA GGCCGGGCCT GACTTTGGCT GGGGGCAGGG |
| 551 | AGGGGGCTAA GGTGACGCAG GTGGCGCCAG CCAGGTGCAC ACCCAATGCC |
| 601 | CATGAGCCCA GACACTGGAC CCTGCATGGA CCATCGCGGA TAGACAAGAA |
| 651 | CCGAGGGGCC TCTGCGCCCT GGGCCCAGCT CTGTCCCACA CCGCGGTCAC |
| 701 | ATGGCACCAC CTCTCTTGCA GCTTCCACCA AGGGCCCATC CGTCTTCCCC |
| 751 | CTGGCGCCCT GCTCCAGGAG CACCTCCGAG AGCACAGCCG CCCTGGGCTG |
| 801 | CCTGGTCAAG GACTACTTCC CCGAACCGGT GACGGTGTCG TGGAACTCAG |
| 851 | GCGCCCTGAC CAGCGGCGTG CACACCTTCC CGGCTGTCCT ACAGTCCTCA |
| 901 | GGACTCTACT CCCTCAGCAG CGTGGTGACC GTGCCCTCCA GCAGCTTGGG |
| 951 | CACGAAGACC TACACCTGCA ACGTAGATCA CAAGCCCAGC AACACCAAGG |
| 1001 | TGGACAAGAG AGTTGGTGAG AGGCCAGCAC AGGGAGGGAG GGTGTCTGCT |
| 1051 | GGAAGCCAGG CTCAGCCCTC CTGCCTGGAC GCACCCCGGC TGTGCAGCCC |
| 1101 | CAGCCCAGGG CAGCAAGGCA TGCCCCATCT GTCTCCTCAC CCGGAGGCCT |
| 1151 | CTGACCACCC CACTCATGCT CAGGGAGAGG GTCTTCTGGA TTTTTCCACC |
| 1201 | AGGCTCCGGG CAGCCACAGG CTGGATGCCC CTACCCCAGG CCCTGCGCAT |
| 1251 | ACAGGGGCAG GTGCTGCGCT CAGACCTGCC AAGAGCCATA TCCGGGAGGA |
| 1301 | CCCTGCCCCT GACCTAAGCC CACCCCAAAG GCCAAACTCT CCACTCCCTC |
| 1351 | AGCTCAGACA CCTTCTCTCC TCCCAGATCT GAGTAACTCC CAATCTTCTC |
| 1401 | TCTGCAGAGT CCAAATATGG TCCCCCATGC CCATCATGCC CAGGTAAGCC |
| 1451 | AACCCAGGCC TCGCCCTCCA GCTCAAGGCG GGACAGGTGC CCTAGAGTAG |
| 1501 | CCTGCATCCA GGGACAGGCC CCAGCCGGGT GCTGACGCAT CCACCTCCAT |
| 1551 | CTCTTCCTCA GCACCTGAGT TCCTGGGGGG ACCATCAGTC TTCCTGTTCC |
| 1601 | CCCCAAAACC CAAGGACACT CTCATGATCT CCCGGACCCC TGAGGTCACG |
| 1651 | TGCGTGGTGG TGGACGTGAG CCAGGAAGAC CCCGAGGTCC AGTTCAACTG |
| 1701 | GTACGTGGAT GGCGTGGAGG TGCATAATGC CAAGACAAAG CCGCGGGAGG |
| 1751 | AGCAGTTCAA CAGCACGTAC CGTGTGGTCA GCGTCCTCAC CGTCCTGCAC |
| 1801 | CAGGACTGGC TGAACGGCAA GGAGTACAAG TGCAAGGTCT CCAACAAAGG |
| 1851 | CCTCCCGTCC TCCATCGAGA AAACCATCTC CAAAGCCAAA GGTGGGACCC |
| 1901 | ACGGGGTGCG AGGGCCACAT GGACAGAGGT CAGCTCGGCC CACCCTCTGC |
| 1951 | CCTGGGAGTG ACCGCTGTGC CAACCTCTGT CCCTACAGGG CAGCCCCGAG |
| 2001 | AGCCACAGGT GTACACCCTG CCCCCATCCC AGGAGGAGAT GACCAAGAAC |
| 2051 | CAGGTCAGCC TGACCTGCCT GGTCAAAGGC TTCTACCCCA GCGACATCGC |
| 2101 | CGTGGAGTGG GAGAGCAATG GGCAGCCGGA GAACAACTAC AAGACCACGC |
| 2151 | CTCCCGTGCT GGACTCCGAC GGCTCCTTCT TCCTCTACAG CAGGCTAACC |
| 2201 | GTGGACAAGA GCAGGTGGCA GGAGGGGAAT GTCTTCTCAT GCTCCGTGAT |
| 2251 | GCATGAGGCT CTGCACAACC ACTACACACA GAAGAGCCTC TCCCTGTCTC |
| 2301 | TGGGTAAATG A |

TABLE 13
TGN2122/TGN2422.C-kappa LC Amino acid sequence

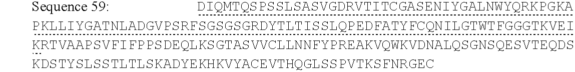

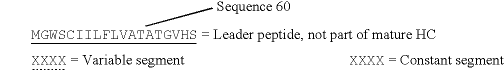

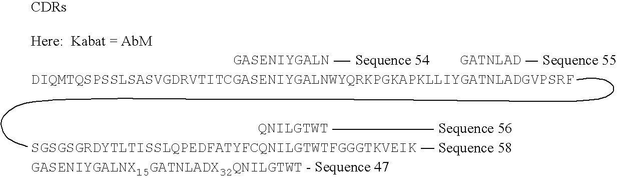

TABLE 14

TGN2122/TGN2422.C-kappa LC Nucleotide sequence

(Sequence 63)

| | |
|---|---|
| 1 | ATGGGATGGA GCTGTATCAT CCTCTTCTTG GTAGCAACAG CTACAGGTAA |
| 51 | GGGGCTCACA GTAGCAGGCT TGAGGTCTGG ACATATATAT GGGTGACAAT |
| 101 | GACATCCACT TTGCCTTTCT CTCCACAGGT GTGCATTCCG ACATCCAGAT |
| 151 | GACCCAGAGC CCCAGCAGCC TGAGCGCCAG CGTGGGCGAC AGGGTGACCA |
| 201 | TCACCTGCGG CGCCAGCGAG AACATCTACG GCGCCCTGAA CTGGTACCAG |
| 251 | AGGAAGCCCG GCAAGGCCCC CAAGCTGCTG ATCTACGGCG CCACCAACCT |
| 301 | GGCCGACGGC GTGCCCAGCA GGTTCAGCGG CAGCGGCAGC GGCAGGGACT |
| 351 | ACACCCTGAC CATCAGCAGC CTGCAGCCCG AGGACTTCGC CACCTACTTC |
| 401 | TGCCAGAACA TCCTGGGCAC CTGGACCTTC GGCGGCGGCA CCAAGGTGGA |
| 451 | GATCAAGCGT GAGTCGTACG CTAGCAAGCT TGATATCGAA TTCTAAACTC |
| 501 | TGAGGGGGTC GGATGACGTG GCCATTCTTT GCCTAAAGCA TTGAGTTTAC |
| 551 | TGCAAGGTCA GAAAAGCATG CAAAGCCCTC AGAATGGCTG CAAAGAGCTC |
| 601 | CAACAAAACA ATTTAGAACT TTATTAAGGA ATAGGGGGAA GCTAGGAAGA |
| 651 | AACTCAAAAC ATCAAGATTT TAAATACGCT TCTTGGTCTC CTTGCTATAA |
| 701 | TTATCTGGGA TAAGCATGCT GTTTTCTGTC TGTCCCTAAC ATGCCCTGTG |
| 751 | ATTATCCGCA AACAACACAC CCAAGGGCAG AACTTTGTTA CTTAAACACC |
| 801 | ATCCTGTTTG CTTCTTTCCT CAGGAACTGT GGCTGCACCA TCTGTCTTCA |
| 851 | TCTTCCCGCC ATCTGATGAG CAGTTGAAAT CTGGAACTGC CTCTGTTGTG |
| 901 | TGCCTGCTGA ATAACTTCTA TCCCAGAGAG GCCAAAGTAC AGTGGAAGGT |
| 951 | GGATAACGCC CTCCAATCGG GTAACTCCCA GGAGAGTGTC ACAGAGCAGG |
| 1001 | ACAGCAAGGA CAGCACCTAC AGCCTCAGCA GCACCCTGAC GCTGAGCAAA |
| 1051 | GCAGACTACG AGAAACACAA AGTCTACGCC TGCGAAGTCA CCCATCAGGG |
| 1101 | CCTGAGCTCG CCCGTCACAA AGAGCTTCAA CAGGGGAGAG TGTTAG |

1. Isolated monoclonal antibody, which is specific and agonistic for CTLA-4, whereby the heavy chain of the antibody contains a sequence selected from the group consisting of (Seq.-ID): “22, 23, 24, 25, 26, 27, 28, 29, 30 and 32”.

2. Isolated monoclonal antibody according to claim 1, whereby the light chain of the antibody contains a sequence selected from the group consisting of (Seq.-ID): “33, 34, 35, 36, 37 and 38”.

3. Isolated monoclonal antibody according to claim 1, with a heavy chain containing a sequence in accordance with Seq.-ID 27, 28 or 29, preferably containing or consisting of the sequence in accordance with Seq.-ID 30 or 32, as well as with a light chain containing a sequence in accordance with Seq.-ID 36 or 37, preferably containing or consisting of a sequence in accordance with Seq.-ID 38.

4. Isolated monoclonal antibody, which is specific and agonistic for CTLA-4, whereby the heavy chain of the antibody contains a sequence selected from the group consisting of (Seq.-ID): “43, 44, 45, 46, 47, 48, 49, 50, 51 and 53”.

5. Isolated monoclonal antibody according to claim 4, whereby the light chain of the antibody contains a sequence selected from the group consisting of (Seq.-ID): “54, 55, 56, 57, 58 and 59”.

6. Isolated monoclonal antibody according to claim 4, with a heavy chain containing a sequence in accordance with Seq.-ID 48, 49, or 50, preferably containing or consisting of the sequence in accordance with Seq.-ID 51 or 53, as well as with a light chain containing a sequence in accordance with Seq.-ID 57 or 58, preferably containing or consisting of a sequence in accordance with Seq.-ID 59.

7. Isolated monoclonal antibody, which is specific and agonistic for CTLA-4, whereby the antibody does not bind to a partial CTLA-4 sequence in accordance with Seq.-ID 1.

8. Isolated monoclonal antibody according to claim 7, containing 1, 2, 3, 4, 5 or 6 of the sequences according to SEQ.-ID 2 through SEQ.-ID 7 or the sequences according to SEQ.-ID 8 through SEQ.-ID 13.

9. Isolated monoclonal antibody according to claim 7, which is humanised.

10. Isolated monoclonal antibody according to claim 7, containing one or both of the sequences according to SEQ.-ID 14 and SEQ.-ID 15 or SEQ.-ID 16 and SEQ.-ID 17, or containing one or both of the sequences according to SEQ.-ID 18 and SEQ.-ID 19 or SEQ.-ID 20 and SEQ.-ID 21.

11. Isolated protein or peptide containing at least one of the sequences SEQ.-ID 2 through 13, in particular one of the sequences SEQ.-ID 14 through 17 or SEQ.-ID 18 through 21, or one of the sequences SEQ.-ID 22, 23, 24, 25, 26, 27, 28, 29, 30, 31, 32, 33, 34, 35, 36, 37, 38 or 39, in particular one of the sequences Seq.-ID 27, 28, 29, 30, 32, 36, 37 or 38, or one of the sequences Seq.- ID 43, 44, 45, 46, 47, 48, 49, 50, 51, 52, 53, 54, 55, 56, 57, 58, 59 or 60, in particular one of the sequences Seq.-ID 48, 49, 50, 51, 53, 57, 58 or 59, or consisting of one of the named sequences.

12. Isolated nucleic acid encoding for a protein or peptide according to claim 11.

13. Isolated vector containing at least one nucleic acid according to claim 12.

14. Isolated cell, whereby the cell is transfixed with the help of a vector according to claim 13.

15. Pharmaceutical compound containing a monoclonal antibody according to claim 1.

16. Use of a monoclonal antibody which is specific and agonistic for CTLA-4, whereby the heavy chain of the antibody contains a sequence selected from the group consisting of (Seq.-ID): “22, 23, 24, 25, 26, 27, 28, 29, 30 and 32”, or a protein or peptide according to claim 11 for producing a pharmaceutical compound for the prophylactic and/or therapeutic treatment of a disease or a condition out of the group comprising “rheumatoid arthritis, type I diabetes, multiple sclerosis, systemic lupus erythematodes, psoriasis, ulcerative colitis, morbus crohn, allergies, rejection of allogenous organ transplants, in particular organ transplants of the following organs: heart, kidney, liver, pancreas, lung, bone marrow, and ‘Graft-Versus-Host’ disease”.

17. Method for producing an isolated monoclonal antibody, which is specific and agonistic for CTLA-4, whereby the heavy chain of the antibody contains a sequence selected from the group consisting of (Seq.-ID): “22, 23, 24, 25, 26, 27, 28, 29, 30 and 32”, whereby a nucleic acid according to claim 12 is incorporated into a vector, whereby a cell is transfixed with the help of the vector, whereby the transfixed cell is cultivated, whereby an excess of the cultivated cell is cut off or whereby the cultivated cell is lysed and the lysate is obtained, and whereby the monoclonal antibodies are separated from the cut off excess or the lysate.

18. Method for producing a pharmaceutical compound according to claim 15, whereby a physiologically effective dosis of the monoclonal antibody and/or the protein or peptide is mixed with at least one physiologically compatible carrier substance and/or agent and prepared in a defined form for administration.

19. Isolated nucleic acid encoding for a light chain and/or heavy chain of an antibody according to claim 1, in particular containing or consisting of a sequence Seq.-ID 61, 62 or 63.

20. Pharmaceutical compound containing a monoclonal antibody according to a protein or peptide according to claim 11, as well as optionally at least one physiologically compatible carrier substance or agent.


SEQUENCE LISTING (63 sequences)

SEQ ID NO: 1 - protein, 6 aa, artificial sequence
  C-D-Loop of Human CD28
LTFLDD

SEQ ID NO: 2 - protein, 10 aa, artificial sequence
  4.3, CDR-H1 of antibody
GYTFTDYKIH

SEQ ID NO: 3 - protein, 17 aa, artificial sequence
  4.3, CDR-H2 of antibody
YIYPYSGSSDYNQKFKS

SEQ ID NO: 4 - protein, 7 aa, artificial sequence
  4.3, CDR-H3 of antibody
GGDAMDY

SEQ ID NO: 5 - protein, 11 aa, artificial sequence
  4.3, CDR-L1 of antibody
GASENIYGALN

SEQ ID NO: 6 - protein, 7 aa, artificial sequence
  4.3, CDR-L2 of antibody
GATNLAD

SEQ ID NO: 7 - protein, 8 aa, artificial sequence
  4.3, CDR-L3 of antibody
QNILGTWT

SEQ ID NO: 8 - protein, 10 aa, artificial sequence
  4.8, CDR-H1 of antibody
GFTFNIYYMS

SEQ ID NO: 9 - protein, 17 aa, artificial sequence
  4.8, CDR-H2 of antibody
AINPDGGNTYYPDTVKG

SEQ ID NO: 10 - protein, 8 aa, artificial sequence
  4.8, CDR-H3 of antibody
YGGPGFDS

SEQ ID NO: 11 - protein, 10 aa, artificial sequence
  4.8, CDR-L1 of antibody
SASSSVSYMH

SEQ ID NO: 12 - protein, 7 aa, artificial sequence
  4.8, CDR-L2 of antibody
DTSKLAS

SEQ ID NO: 13 - protein, 11 aa, artificial sequence
  4.8, CDR-L3 of antibody
FPGSGFPFMYT

SEQ ID NO: 14 - protein, 116 aa, artificial sequence
  4.3, heavy chain of antibody
XXXXXXXXXXXXXXXXXXXXXXXXXGYTFTDYKIHXXXXXXXXXXXXXXYIYPYSGSSDYNQKFKSXXXXXXXXXXXXXXXXXXXXXXXXXXXXXXXXGGDAMDYXXXXXXXXXXX

SEQ ID NO: 15 - protein, 107 aa, artificial sequence
  4.3, light chain of antibody
XXXXXXXXXXXXXXXXXXXXXXXGASENIYGALNXXXXXXXXXXXXXXXGATNLADXXXXXXXXXXXXXXXXXXXXXXXXXXXXXXXXQNILGTWTXXXXXXXXXXX

SEQ ID NO: 16 - protein, 117 aa, artificial sequence
  4.8, heavy chain of antibody
XXXXXXXXXXXXXXXXXXXXXXXXXGFTFNIYYMSXXXXXXXXXXXXXXAINPDGGNTYYPDTVKGXXXXXXXXXXXXXXXXXXXXXXXXXXXXXXXXYGGPGFDSXXXXXXXXXXX

SEQ ID NO: 17 - protein, 109 aa, artificial sequence
  4.8, light chain of antibody
XXXXXXXXXXXXXXXXXXXXXXXSASSSVSYMHXXXXXXXXXXXXXXXDTSKLASXXXXXXXXXXXXXXXXXXXXXXXXXXXXXXXXFPGSGFPFMYTXXXXXXXXXXX

SEQ ID NO: 18 - protein, 116 aa, artificial sequence
  4.3, heavy chain of antibody
EVQLQPSGPVLVKPGASVKISCKASGYTFTDYKIHWVKQSHGKSLEWIGYIYPYSGSSDYNQKFKSKATLTVDNSSTTAYMELRSLTSEDSAVYYCARGGDAMDYWGQGTAVTVSS

SEQ ID NO: 19 - protein, 107 aa, artificial sequence
  4.3, light chain of antibody
DILMTQSPASLSASVGETVTITCGASENIYGALNWYQRKQGKSPQLLIYGATNLADGMSSRFSGSGLGRQYYLKISSLHPDDVATYFCQNILGTWTFGGGTKLEIKR

SEQ ID NO: 20 - protein, 117 aa, artificial sequence
  4.8, heavy chain of antibody
DVKLVESGGGLVKLGGSLKLSCAASGFTFNIYYMSWVRQTPEKRLELVAAINPDGGNTYYPDTVKGRFTISRDNAKNTLYLQMSSLKSEDSALYYCARYGGPGFDSWGQGTTLTVSS

SEQ ID NO: 21 - protein, 109 aa, artificial sequence
  4.8, light chain of antibody
ENVLTQSPAIMSASPGERVTMTCSASSSVSYMHWYQQKSNTSPKLWIYDTSKLASGVPGRFSGSGSRNSYSLTISSMEAEDVATYYCFPGSGFPFMYTFGGGTKLEIKR

SEQ ID NO: 22 - protein, 5 aa, artificial sequence
  partial sequence of antibody
IYYMS

SEQ ID NO: 23 - protein, 17 aa, artificial sequence
  partial sequence of antibody
AINPDGGNTYYPDTVKG

SEQ ID NO: 24 - protein, 8 aa, artificial sequence
  partial sequence of antibody
YGGPGFDS

SEQ ID NO: 25 - protein, 10 aa, artificial sequence
  partial sequence of antibody
AINPDGGNTY

SEQ ID NO: 26 - protein, 10 aa, artificial sequence
  partial sequence of antibody
AINPDGGNTY

SEQ ID NO: 27 - protein, 117 aa, artificial sequence
  partial sequence of antibody
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSS

SEQ ID NO: 28 - protein, 76 aa, artificial sequence
  partial sequence of antibody
IYYMSXXXXXXXXXXXXXXAINPDGGNTYYPDTVKGXXXXXXXXXXXXXXXXXXXXXXXXXXXXXXXXYGGPGFDS

SEQ ID NO: 29 - protein, 81 aa, artificial sequence
  partial sequence of antibody
GFTFNIYYMSXXXXXXXXXXXXXXAINPDGGNTYXXXXXXXXXXXXXXXXXXXXXXXXXXXXXXXXXXXXXXXYGGPGFDS

SEQ ID NO: 30 - protein, 447 aa, artificial sequence
  partial sequence of antibody
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 19 aa, artificial sequence
  partial sequence of antibody
MGWSCIILFLVATATGVHS

SEQ ID NO: 32 - protein, 444 aa, artificial sequence
  partial sequence of antibody
EVQLVESGGGLVQPGGSLRLSCAASGFTFNIYYMSWVRQAPGKGLELVAAINPDGGNTYYPDTVKGRFTISRDNAKNSLYLQMNSLRAEDTAVYYCARYGGPGFDSWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 10 aa, artificial sequence
  partial sequence of antibody
SASSSVSYMH

SEQ ID NO: 34 - protein, 7 aa, artificial sequence
  partial sequence of antibody
DTSKLAS

SEQ ID NO: 35 - protein, 11 aa, artificial sequence
  partial sequence of antibody
FPGSGFPFMYT

SEQ ID NO: 36 - protein, 75 aa, artificial sequence
  partial sequence of antibody
SASSSVSYMHXXXXXXXXXXXXXXXDTSKLASXXXXXXXXXXXXXXXXXXXXXXXXXXXXXXXXFPGSGFPFMYT

SEQ ID NO: 37 - protein, 108 aa, artificial sequence
  partial sequence of antibody
ENVLTQSPATLSLSPGERATLSCSASSSVSYMHWYQQKPGQAPRLWIYDTSKLASGIPARFSGSGSRNDYTLTISSLEPEDFAVYYCFPGSGFPFMYTFGGGTKVEIK

SEQ ID NO: 38 - protein, 215 aa, artificial sequence
  partial sequence of antibody
ENVLTQSPATLSLSPGERATLSCSASSSVSYMHWYQQKPGQAPRLWIYDTSKLASGIPARFSGSGSRNDYTLTISSLEPEDFAVYYCFPGSGFPFMYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 19 aa, artificial sequence
  partial sequence of antibody
MGWSCIILFLVATATGVHS

SEQ ID NO: 40 - DNA, 2317 bp, artificial sequence
  partial sequence coding for antibody fragment
atgggatggagctgtatcatcctcttcttggtagcaacagctacaggtaaggggctcacagtagcaggcttgaggtctggacatatatatgggtgacaatgacatccactttgcctttctctccacaggtgtgcattccgaggtgcagctggtggagagcggcggcggcctggtgcagcccggcggcagcctgaggctgagctgcgccgccagcggcttcaccttcaacatctactacatgagctgggtgaggcaggcccccggcaagggcctggagctggtggccgccatcaaccccgacggcggcaacacctactaccccgacaccgtgaagggcaggttcaccatcagcagggacaacgccaagaacagcctgtacctgcagatgaacagcctgagggccgaggacaccgccgtgtactactgcgccaggtacggcggccccggcttcgacagctggggccagggcaccctggtgaccgtgagcagcggtgagtcgtacgctagcaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 41 - DNA, 2314 bp, artificial sequence
  partial sequence coding for antibody fragment
atgggatggagctgtatcatcctcttcttggtagcaacagctacaggtaaggggctcacagtagcaggcttgaggtctggacatatatatgggtgacaatgacatccactttgcctttctctccacaggtgtgcattccgaggtgcagctggtggagagcggcggcggcctggtgcagcccggcggcagcctgaggctgagctgcgccgccagcggcttcaccttcaacatctactacatgagctgggtgaggcaggcccccggcaagggcctggagctggtggccgccatcaaccccgacggcggcaacacctactaccccgacaccgtgaagggcaggttcaccatcagcagggacaacgccaagaacagcctgtacctgcagatgaacagcctgagggccgaggacaccgccgtgtactactgcgccaggtacggcggccccggcttcgacagctggggccagggcaccctggtgaccgtgagcagcggtgagtcgtacgctagcaagctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 42 - DNA, 1152 bp, artificial sequence
  partial sequence coding for antibody fragment
atgggatggagctgtatcatcctcttcttggtagcaacagctacaggtaaggggctcacagtagcaggcttgaggtctggacatatatatgggtgacaatgacatccactttgcctttctctccacaggtgtgcattccgagaacgtgctgacccagagccccgccaccctgagcctgagccccggcgagagggccaccctgagctgcagcgccagcagcagcgtgagctacatgcactggtaccagcagaagcccggccaggcccccaggctgtggatctacgacaccagcaagctggccagcggcatccccgccaggttcagcggcagcggcagcaggaacgactacaccctgaccatcagcagcctggagcccgaggacttcgccgtgtactactgcttccccggcagcggcttccccttcatgtacaccttcggcggcggcaccaaggtggagatcaagcgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 43 - protein, 5 aa, artificial sequence
  partial sequence of antibody
DYKIH

SEQ ID NO: 44 - protein, 17 aa, artificial sequence
  partial sequence of antibody
YIYPYSGSSDYNQKFKS

SEQ ID NO: 45 - protein, 7 aa, artificial sequence
  partial sequence of antibody
GGDAMDY

SEQ ID NO: 46 - protein, 10 aa, artificial sequence
  partial sequence of antibody
GYTFTDYKIH

SEQ ID NO: 47 - protein, 10 aa, artificial sequence
  partial sequence of antibody
YIYPYSGSSD

SEQ ID NO: 48 - protein, 116 aa, artificial sequence
  partial sequence of antibody
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSS

SEQ ID NO: 49 - protein, 75 aa, artificial sequence
  partial sequence of antibody
DYKIHXXXXXXXXXXXXXXYIYPYSGSSDYNQKFKSXXXXXXXXXXXXXXXXXXXXXXXXXXXXXXXXGGDAMDY

SEQ ID NO: 50 - protein, 80 aa, artificial sequence
  partial sequence of antibody
GYTFTDYKIHXXXXXXXXXXXXXXYIYPYSGSSDXXXXXXXXXXXXXXXXXXXXXXXXXXXXXXXXXXXXXXXGGDAMDY

SEQ ID NO: 51 - protein, 446 aa, artificial sequence
  partial sequence of antibody
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 19 aa, artificial sequence
  partial sequence of antibody
MGWSCIILFLVATATGVHS

SEQ ID NO: 53 - protein, 443 aa, artificial sequence
  partial sequence of antibody
QVQLVQSGAEVKKPGASVKVSCKASGYTFTDYKIHWVRQAPGQGLEWIGYIYPYSGSSDYNQKFKSRATLTVDNSISTAYMELSRLRSDDTAVYYCARGGDAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 11 aa, artificial sequence
  partial sequence of antibody
GASENIYGALN

SEQ ID NO: 55 - protein, 7 aa, artificial sequence
  partial sequence of antibody
GATNLAD

SEQ ID NO: 56 - protein, 8 aa, artificial sequence
  partial sequence of antibody
QNILGTWT

SEQ ID NO: 57 - protein, 73 aa, artificial sequence
  partial sequence of antibody
GASENIYGALNXXXXXXXXXXXXXXXGATNLADXXXXXXXXXXXXXXXXXXXXXXXXXXXXXXXXQNILGTWT

SEQ ID NO: 58 - protein, 106 aa, artificial sequence
  partial sequence of antibody
DIQMTQSPSSLSASVGDRVTITCGASENIYGALNWYQRKPGKAPKLLIYGATNLADGVPSRFSGSGSGRDYTLTISSLQPEDFATYFCQNILGTWTFGGGTKVEIK

SEQ ID NO: 59 - protein, 213 aa, artificial sequence
  partial sequence of antibody
DIQMTQSPSSLSASVGDRVTITCGASENIYGALNWYQRKPGKAPKLLIYGATNLADGVPSRFSGSGSGRDYTLTISSLQPEDFATYFCQNILGTWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 19 aa, artificial sequence
  partial sequence of antibody
MGWSCIILFLVATATGVHS

SEQ ID NO: 61 - DNA, 2314 bp, artificial sequence
  partial sequence coding for antibody fragment
atgggatggagctgtatcatcctcttcttggtagcaacagctacaggtaaggggctcacagtagcaggcttgaggtctggacatatatatgggtgacaatgacatccactttgcctttctctccacaggtgtgcattcccaggtgcagctggtgcagagcggcgccgaggtgaagaagcccggcgccagcgtgaaggtgagctgcaaggccagcggctacaccttcaccgactacaagatccactgggtgaggcaggcccccggccagggcctggagtggatcggctacatctacccctacagcggcagcagcgactacaaccagaagttcaagagcagggccaccctgaccgtggacaacagcatcagcaccgcctacatggagctgagcaggctgaggagcgacgacaccgccgtgtactactgcgccaggggcggcgacgccatggactactggggccagggcaccctggtgaccgtgagcagcggtgagtcgtacgctagcaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 62 - DNA, 2311 bp, artificial sequence
  partial sequence coding for antibody fragment
atgggatggagctgtatcatcctcttcttggtagcaacagctacaggtaaggggctcacagtagcaggcttgaggtctggacatatatatgggtgacaatgacatccactttgcctttctctccacaggtgtgcattcccaggtgcagctggtgcagagcggcgccgaggtgaagaagcccggcgccagcgtgaaggtgagctgcaaggccagcggctacaccttcaccgactacaagatccactgggtgaggcaggcccccggccagggcctggagtggatcggctacatctacccctacagcggcagcagcgactacaaccagaagttcaagagcagggccaccctgaccgtggacaacagcatcagcaccgcctacatggagctgagcaggctgaggagcgacgacaccgccgtgtactactgcgccaggggcggcgacgccatggactactggggccagggcaccctggtgaccgtgagcagcggtgagtcgtacgctagcaagctttctggggcaggccgggcctgactttggctgggggcagggagggggctaaggtgacgcaggtggcgccagccaggtgcacacccaatgcccatgagcccagacactggaccctgcatggaccatcgcggatagacaagaaccgaggggcctctgcgccctgggcccagctctgtcccacaccgcggtcacatggcaccacctctcttgcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttggtgagaggccagcacagggagggagggtgtctgctggaagccaggctcagccctcctgcctggacgcaccccggctgtgcagccccagcccagggcagcaaggcatgccccatctgtctcctcacccggaggcctctgaccaccccactcatgctcagggagagggtcttctggatttttccaccaggctccgggcagccacaggctggatgcccctaccccaggccctgcgcatacaggggcaggtgctgcgctcagacctgccaagagccatatccgggaggaccctgcccctgacctaagcccaccccaaaggccaaactctccactccctcagctcagacaccttctctcctcccagatctgagtaactcccaatcttctctctgcagagtccaaatatggtcccccatgcccatcatgcccaggtaagccaacccaggcctcgccctccagctcaaggcgggacaggtgccctagagtagcctgcatccagggacaggccccagccgggtgctgacgcatccacctccatctcttcctcagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaaggtgggacccacggggtgcgagggccacatggacagaggtcagctcggcccaccctctgccctgggagtgaccgctgtgccaacctctgtccctacagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 63 - DNA, 1146 bp, artificial sequence
  partial sequence coding for antibody fragment
atgggatggagctgtatcatcctcttcttggtagcaacagctacaggtaaggggctcacagtagcaggcttgaggtctggacatatatatgggtgacaatgacatccactttgcctttctctccacaggtgtgcattccgacatccagatgacccagagccccagcagcctgagcgccagcgtgggcgacagggtgaccatcacctgcggcgccagcgagaacatctacggcgccctgaactggtaccagaggaagcccggcaaggcccccaagctgctgatctacggcgccaccaacctggccgacggcgtgcccagcaggttcagcggcagcggcagcggcagggactacaccctgaccatcagcagcctgcagcccgaggacttcgccacctacttctgccagaacatcctgggcacctggaccttcggcggcggcaccaaggtggagatcaagcgtgagtcgtacgctagcaagcttgatatcgaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag